(19) United States
(12) Patent Application Publication
(10) Pub. No.: US 2015/0051166 A1
(43) Pub. Date: Feb. 19, 2015

(54) Methods of Treating Neurological Diseases

(71) Applicants: Jokubas Ziburkus, Houston, TX (US); Jason Eriksen, Houston, TX (US); Feng Gu, Houston, TX (US)

(72) Inventors: Jokubas Ziburkus, Houston, TX (US); Jason Eriksen, Houston, TX (US); Feng Gu, Houston, TX (US)

(21) Appl. No.: 13/969,252

(22) Filed: Aug. 16, 2013

(51) Int. Cl. A61K 31/7076; A61K 45/06

(52) U.S. Cl. CPC A61K 31/7076; A61K 45/06
USPC 514/46

(57) ABSTRACT

The present invention is directed to novel treatment of controlling hippocampal neural circuit hyperexcitability occurring in a neurological disease or disorder associated with epileptogenesis in a subject in need of such treatment, comprising the step of contacting the hippocampus in said subject with a compound effective to restore excitatory/inhibitor balance thereby controlling the neural circuit hyperexcitability. Further provided is a method of treating a neurological disease or disorder associated with epileptogenesis in a subject in need of such treatment, comprising the step of administering an amount of an adenosine A1 receptor agonist pharmacologically effective to block epileptogenetic activities without blocking excitatory synaptic transmission.

### CROSS-REFERENCE TO RELATED APPLICATIONS

This application is a non-provisional under 35 U.S.C. 119(e) of provisional application U.S. Ser. No. 61/684,213, filed Aug. 17, 2012, now abandoned, the entirety of which is hereby incorporated by reference.

### BACKGROUND OF THE INVENTION

1. Field of the Invention

The present invention is in the field of pharmacotherapy of neurological diseases. More specifically, the present invention is directed to novel treatment of neurological diseases via manipulation of neural adenosine activity.

2. Description of the Related Art

Severe myoclonic epilepsy in infancy (SMEI) or Dravet syndrome is one of the most deleterious forms of childhood epilepsy, with onset in the first year of life, usually beginning with febrile seizures (1). These generalized seizures can often culminate into status epilepticus and SMEI patients often suffer from a number of devastating neurological complications (2-7).

Genetic studies show that 70-80% of SMEI phenotype can be accounted for by mutations in the SCN1A gene. Several recent studies confirm that multiple SCN1A gene mutations, which affect voltage-gated sodium channel protein (Nav1.1), lead to epilepsy phenotypes that strikingly recapitulate many phenotypes of the human SMEI disorder, like low threshold for febrile seizures and early death in homozygous mutants (1, 8-11). On a cellular level, studies of the mSMEI have also demonstrated that this mutation is identical to the mutations found in three unrelated patients with SMEI (12). When the mutated Naᵥ1.1 channels were expressed in cultured cells, the sodium currents were significantly reduced (13). Later, it was shown that this and similar SCN1A mutations specifically affect neocortical (14) and hippocampal (15) inhibitory interneurons, causing them to fail to reliably generate action potentials.

Despite recent advances in understanding the pathophysiology of SMEI, effective treatments for it still remain a great challenge (16-17). SMEI patients are clinically refractory with 10-20% mortality rate. To improve knowledge of epileptogenesis in mSMEI, further studies are needed to elucidate the impact of SCN1A mutations on synaptic and circuit pathophysiology.

Recent advances in fast functional imaging, including voltage-sensitive dye imaging (VSDI) provide a way to simultaneously measure the membrane potential of neuronal populations across wide spatial areas, enabling identification of hyperexcitable circuits. VSDI signals are linearly correlated with postsynaptic neuronal membrane potential fluctuations (18-20) and can be used reliably to visualize evoked (21-22) or spontaneous epileptiform activity (23). In chronic epilepsy models, VSDI reveals circuit hyperexcitability, synonymous with significantly wider area of evoked neural activation. This approach, however, has not been applied to study the neural circuits mediating pathophysiology in mSMEI.

The prior art is deficient in the lack of effective treatments of neurological diseases such as intractable epilepsy, Dravet syndrome, febrile seizures, autism spectrum disorders and attention deficit hyperactivity disorders. The present invention fulfills this longstanding need and desire in the art.

### SUMMARY OF THE INVENTION

The present invention is directed to a method of controlling hippocampal neural circuit hyperexcitability occurring in a neurological disease or disorder associated with epileptogenesis in a subject. The method comprises contacting the hippocampus in the subject with a compound effective to restore excitatory/inhibitory (E/I) balance thereby controlling the neural circuit hyperexcitability. The compound may be, but is not limited to, adenosine, an adenosine mimetic, an adenosine modulator, an adenosine transport inhibitor, or an adenosine receptor agonist.

The present invention also is directed to a method of treating a neurological disease or disorder associated with epileptogenesis. The method comprises administering one or more times an amount of an adenosine A1 receptor agonist pharmacologically effective to block epileptogenetic activities without blocking excitatory synaptic transmission. Representative examples of adenosine A1 receptor agonists are adenosine receptor congeners, N6-cyclopentyladenosine; N6-cyclohexyladenosine; 2-chloro-cyclopentyladenosine; N-(3(R))-tetrahydrofuranyl)-6-aminopurine riboside; or nucleoside transporters. Representative examples of neurological diseases and disorders are intractable epilepsy, Dravet syndrome, febrile seizures, autism spectrum disorders and attention deficit hyperactivity disorders.

The present invention is directed further to a method of treating severe myoclonic epilepsy in infancy in a subject. The method comprises administering one or more times to the subject an amount of N6-cyclopentyladenosine, thereby treating the myoclonic epilepsy.

### BRIEF DESCRIPTION OF THE DRAWINGS

The appended drawings have been included herein so that the above-recited features, advantages and objects of the invention will become clear and can be understood in detail. These drawings form a part of the specification. It is to be noted, however, that the appended drawings illustrate preferred embodiments of the invention and should not be considered to limit the scope of the invention.

FIGS. 1A-1E show increased excitation in the CA1 of mSMEI. FIG. 1A: Evoked fEPSP amplitude measurements, showing that incremental increases in the stimulation currents of Schaeffer collaterals elicited significantly larger responses in the HET hippocampi area CA1 (N=5; P=0.0008, unpaired t test). FIGS. 1B-1C: Electrical traces of sEPSCs from the pyramidal cells recorded in HET and WT tissue. Downward deflections in the electrical traces are spontaneous inward excitatory currents or sEPSCs. FIG. 1D: Cumulative distribution plot of the inter-event interval duration or frequency of occurrence of sEPSCs (N=6; 760.9±28.47 vs. 800.9±43.99, p=0.4415, unpaired t test). FIG. 1E: Cumulative distribution plot of the sEPSC amplitudes. Synaptic excitation was increased in the HET animal tissue, as indicated by the rightward shift of the amplitude plot. **indicates statistical significance (N=6; 17.28±0.3533 vs. 13.64±0.3056, p<0.0001, unpaired t test).

FIGS. 2A-2D shows decreased IPSCs in SCN1A mutants. FIGS. 2A-2B: Whole-cell voltage-clamp traces from CA1 pyramidal cells held at −80 mV. Recordings were obtained in the presence of glutamatergic transmission blockers CNQX (40 μM) and APV (100 μM). Arrows indicated a portion of expanded traces on the right. FIGS. 2C-2D: Cumulative distribution of sIPSC interval and amplitudes. Both, frequency (FIG. 2C) and amplitudes (FIG. 2D) of the sIPSCs were significantly reduced in the HET pyramidal cells. (c: N=8 HET, 5 WT; 180.7±3.475 vs. 53.52±1.058, p<0.0001, unpaired t test; d: N=8 HET, 5 WT; 15.59±0.3702 vs. 22.57±0.3761, p<0.0001, unpaired t-test).

FIGS. 3A-3D show impaired synaptic plasticity in mSMEI. FIGS. 3A-3B: Representative electrical traces of fEPSPs evoked by 40 Hz stimulation train in WT (FIG. 3A) and HET (FIG. 3B) hippocampus area CA1. FIG. 3C: Average fEPSP amplitudes in WT and HET tissue during the 40 Hz stimulation. fEPSPs were significantly larger in the HET tissue (N=6 slices in WT and in HET conditions, p<0.0001). FIG. 3D: STP plots were produced by comparing individual amplitude of pulses #2-10 to the amplitude of pulse #1. Divergence in the amount of facilitation can be best observed in the later portion of the fEPSP train responses. fEPSP ratios from the two measured populations showed significant differences in the degree of STP (N=6 slices each, HET and WT, p=0.0003, unpaired t-test).

FIGS. 4A-4G show increased propagation of neural activity in mSMEI hippocampal circuits. FIGS. 4A, 4C: Photomicrographs depict transverse slices of the hippocampus overlaid with the normalized average (15 trials) VSD signals in WT (FIG. 4A) and HET (FIG. 4C) tissue. Thick black line is the stimulating electrode (200 μm tip) and the site of Schaeffer collateral stimulation in CA1 area. Each frame corresponds to the peak of the signal produced for each of 10 stimulation pulses (P1-P10). v40 Hz train stimulation in the wild type (WT) tissue evoked a typically small and concise neuronal activity map. The same intensity stimulation in the SMEI hippocampi activated widespread neural activity propagation. vNote antidromic signal activation in the CA3 area of HET hippocampus. Scale bar=250 μm. FIGS. 4B, 4D: Optical traces of 40 Hz stimulation in WT (FIG. 4B) and HET (FIG. 4D) tissue from a representative pixel (*) in CA1 region. FIG. 4E: Example of propagation distance calculation using FWHM. To analyze the average distance of the propagated signal, we used a 950 μm long line (indicated by the black arrow) that crossed through the approximate center region of the evoked signal. Scale bar=200 μm. FIG. 4F: Evoked signal over the evoked propagating signal (black arrow in FIG. 4E) is shown for 15 frames before and 94 frames after the 40 Hz stimulation. FIG. 4G: FWHM measurements of the propagating signal show that in HET tissue the signal evoked by the stimulation propagated significantly further distances, toward the subicullum region as compared with the WT responses (HET: 640.5±75.08 μm, N=8; WT: 443.0±33.80 μm, N=9; p=0.0247, unpaired t-test).

FIGS. 5A-5F shows that A1R agonist reliably controls synaptic and circuit hyperexcitability in mSMEI. FIG. 5A: Electrical traces of the evoked potentials recorded extracellularly. In this representative example, very low stimulation intensity (100 μA) evoked population spike (bottom trace). Addition of 50 nM N6-cyclopentyladenosine reduced this spike into a fEPSP response. FIG. 5B: Pharmacological manipulation of fEPSP response amplitudes with A1R agonist CPA and antagonist DPCPX. Example traces of the responses recorded in the CA1 during the train stimulation in: 1) regular ACSF; 2) ACSF with 50 nM CPA; 3) ACSF with N6-cyclopentyladenosine and DPCPX. DPCPX prevented N6-cyclopentyladenosine to decrease fEPSP amplitude. FIG. 5C: Average fEPSP measurements in WT (n=6), HET (n=6), and HET tissue after addition of N6-cyclopentyladenosine (n=6) during ten pulse 40 Hz stimulation pulses. 50 nM N6-cyclopentyladenosine significantly reduced fEPSP trains in the HET hippocampus (paired t-test; p<0.0001). FIGS. 5D-5E: Neural activity map (dF/Fmax) in HET animal evoked at P19. 50 nM of N6-cyclopentyladenosine reduced the abnormally wide circuit excitation. Scale bar (white line) −200 μm. FIG. 5F: N6-cyclopentyladenosine significantly reduced the spatial extent of neural signal propagation (N=6, paired t-test, p<0.0001). FWHM measurements showed significant reduction in the spread of the evoked activity in the presence of N6-cyclopentyladenosine (HET: 591±106 μm; HET+CPA: 491±106 μm; p=0.0321; N=6 slices).

FIGS. 6A-6E shows the mechanics of (FSLE) dynamics. FIGS. 6A-6C: Whole-cell and extracellular DC mode traces of a representative febrile seizure-like event (FSLE). FIGS. 6A-6B: the expanded traces from FIG. 6C. FIG. 6C: Organized activity of FSLEs emerge and terminate as sub-threshold bursts. FSLE was formed at 39° C. in HET. FIG. 6D: Electrical traces of spontaneous IPSCs recorded at 32° C. FIG. 6E: With increasing temperature, sIPSCs are gradually diminished. Electrical traces from the cell after the temperature has been raised to 40° C.

FIGS. 7A-7C shows that SCN1A have lowered threshold for FSLEs. FIG. 7A: Bar graph of the seizure incidence in heterozygote (HET) and wild-type (WT) hippocampal slices. n-total number of slices (with and without FSLEs). FIG. 7B: FSLEs emerged at an average of 38.5° C. in HET (n=20) and 40.5° C. in WT (n=8) tissue. FIG. 7C: FSLEs in the HET tissue (n=25) were almost twice longer in duration as compared to the WT. Seizure duration was measured from the start of the ictal-like event to the re-polarization of the extracellular recordings.

FIGS. 8A-8F shows that CPA reliably controls synaptic and circuit hyperexcitability. Example traces of the evoked potentials recorded extracellularly. FIG. 8A: very low stimulation intensity (100 mA) evoked population spikes (bottom trace). Addition of 50 nM CPA reduced this spike into a field EPSP response. FIG. 8B: Average fEPSP measurements in WT (n=6), HET (n=6), and HET tissue after addition of CPA (n=6) during ten pulse 40 Hz stimulation pulses. 50 nM CPA significantly reduced fEPSP in HET hippocampus. Analysis of the variances (ANOVA) showed that all three groups WT, HET, and HET+CPA were statistically significant from each other (p<0.05, dF=26, Neuman Keuls). FIGS. 8C-8D: Neural activity map (dF/Fmax) in HET animal evoked at P19. 50 nM of CPA reduced the abnormally wide circuit excitation. Scale bar −200 μm. FIG. 8E: CPA significantly reduced the spatial extent of neural signal propagation (N=6, paired t-test, p<0.0001). FIG. 8F: Effects of CPA on short-term plasticity (STP). STP plots were produced by comparing the amplitude of pulses #2-10 to the amplitude of p#1. STP plot difference are best seen in the later portion of the train stimulation. ANOVA showed that all three groups (WT, HET, and HET+CPA (N=6 in each group) were all statistically different, p<0.05; HET vs HET+CPA paired t-test, N=6, p=0.003).

FIGS. 9A-9B show that CPA blocks epileptogenic activity in hyperthermia. FIG. 9A: unfiltered extracellular recording traces (DC mode) of the typical repeated FSLEs in P18 isolated mouse hippocampus. FIG. 9B: unfiltered extracellular trace shows the formation of the first FSLE. Immediately after that CPA was added. Minimal bursting activity was observed, but no repetitive FSLEs formed.

FIGS. 10A-10D show that SCN1A mutants have lower hyperthermia FS threshold. FIG. 10A: In vivo seizure incidence. All 5 HET and 1 in 5 WT animals had FS behavior on Racine scale of 6. FIG. 10B: FS occurred at shorter latency after hyperthermia, and (FIG. 10C:) were longer duration than in one WT animal that had a seizure. FIG. 10D: Photo frames of the typical hyperthermia FS behavior in SCN1A mutants.

FIGS. 11A-11C: Acute CPA treatment suppressed hyperthermia-induced seizure in mSMEI in vivo. FIG. 11A: CPA reduced the seizure incidence when injected intraperitoneally 15 minutes prior to the seizure induction. FIG. 11B: CPA significantly increased the seizure latency (HET+saline: 8.19±0.95, n=7; HET+CPA: 11.68±0.70, n=5; p=0.02, unpaired t test), and FIG. 11C: decreased the seizure duration (HET+saline: 5.91±1.17, n=7; HET+CPA: 2.26±0.43, n=5; p=0.03, unpaired t test).

FIGS. 12A-12C: Effect of chronic CPA treatment on hyperthermia-induced seizure in vivo 24 hours after the last treatment. FIG. 12A: Compared with the vehicle (0.9% saline) group, chronic CPA treatment (P11-P20) reduced the seizure incidence (HET+vehicle: 83.3%, n=17; HET+CPA: 53%, n=6). FIG. 12B: CPA tended to increase the seizure latency, although the difference is not significant. (HET+vehicle: 8.080±1.772, n=9; HET+CPA: 11.25±1.140, n=5; p=0.1413, unpaired t test). FIG. 12C: CPA significantly decreased the seizure duration. (HET+vehicle: 3.980±0.6651, n=9, n=9; HET+CPA: 1.424±0.6904, n=5; p=0.0326, unpaired t test).

FIGS. 13A-13C: Effect of chronic CPA treatment on hyperthermia-induced seizure in vivo 10 days after the last treatment. FIG. 13A: Chronic CPA treatment reduced the seizure incidence (HET+vehicle: 75%, n=12; HET+CPA: 43%, n=7), compared with the vehicle group. FIG. 13B: CPA tended to increase the seizure latency (HET+vehicle: 9.181±1.1.416, n=9; FIG. 13C: HET+CPA: 11.87±1.866, n=3; p=0.3074, unpaired t test) and decrease the duration (HET+vehicle: 10.52±0.9641, n=7; HET+CPA: 6.777±3.597, n=3; p=0.1929, unpaired t test), although the differences are not significant between CPA and vehicle groups.

FIGS. 14A-14D Effect of repeated CPA treatment on inhibition and excitation. FIGS. 14A-14B: Cumulative distribution plot of sEPSC inter-event interval and amplitude show that the interval is decreased and the amplitude is increased after the repeated treatment with CPA, as indicated by the rightward shift of the interval and the leftward shift of amplitude respectively. (n=8 HET+CPA, 5 HET+vehicle; p<0.0001, K-S test). FIGS. 14C-14D: Cumulative distribution plot of sIPSC interval and amplitude show no significant difference between CPA treated group and vehicle treated group. (n=7 HET+CPA, 5 HET+vehicle; K-S test).

### DETAILED DESCRIPTION OF THE PRESENT INVENTION

Severe myoclonic epilepsy in infancy (SMEI) or Dravet syndrome is one of the most devastating childhood epilepsies. Children with SMEI suffer from febrile and afebrile seizures, ataxia, and social and cognitive dysfunctions. SMEI is pharmacologically intractable and can be fatal in 10-20% of patients. Remarkably, genetic mouse models with mutations in the SCN1A gene replicate many aspects of human SMEI. Several recent studies of mouse models of SMEI (mSMEI) with SCN1A gene mutations have elucidated molecular and cellular mechanisms that may account for the epileptogenesis. There remains, however, a critical need to further elucidate how chanellopathies causing SMEI and other epilepsies impact synaptic excitation/inhibition (E/I) balance and neuronal activity in key anatomical circuits.

The purpose of this invention is to analyze and control neural circuit excitability in the developing hippocampus of mSMEI caused by a mutation in the SCN1A gene. Synaptic E/I balance, plasticity, and neural activity propagation characteristics were studied using a combination of electrophysiology and fast voltage-sensitive dye imaging (VSDI) in hippocampal area CA1 in vitro during postnatal days P16-P22. Using whole-cell voltage-clamp recordings we analyzed spontaneous excitatory and inhibitory postsynaptic currents in CA1 pyramidal cells. CA1 circuit activity was studied with a combination of concurrent extracellular recordings and fast VSDI. Field excitatory-postsynaptic potentials (fEPSPs) were evoked along the Schaeffer collateral pathway, projecting from area CA3 into the CA1.

To investigate synaptic excitability and short-term plasticity (STP), single pulse and 40 Hz ten pulse train stimulations were used. To control hippocampal hyperexcitability and abnormally widespread network activation, the adenosine A1 receptor (A1R) agonist N6-cyclopentyladenosine (CPA) was used. The present invention reveals significant E/I imbalance in the mSMEI, showing decreased inhibition, increased excitation, and abnormally wide-spread activity propagation in the CA1 circuit. CPA significantly reduced hippocampal circuit hyperexcitability without blocking excitatory synaptic transmission. These findings fill a gap in the knowledge of synaptic and circuit activity in mSMEI. Results with A1R agonist CPA suggest that this compound reliably controls hippocampal hyperexcitability and warrant its further investigations in mSMEI in vivo.

The articles “a” and “an” are used herein to refer to one or to more than one (i.e. to at least one) of the grammatical object of the article. By way of example, “an element” means one element or more than one element.

The terms “comprise” and “comprising” are used in the inclusive, open sense, meaning that additional elements may be included.

The term “including” is used herein to mean “including but not limited to”. “Including” and “including but not limited to” are used interchangeably.

The term “mammal” is known in the art, and exemplary mammals include humans, primates, bovines, porcines, canines, felines, and rodents (e.g., mice and rats).

A “patient,” “subject” or “host” to be treated by the subject method may mean either a human or non-human mammal.

The term “pharmaceutically-acceptable salts” is art-recognized and refers to the relatively non-toxic, inorganic and organic acid addition salts of compounds, including, for example, those contained in compositions of the present invention.

The term “prodrug” is art-recognized and is intended to encompass compounds which, under physiological conditions, are converted into the antibacterial agents of the present invention. A common method for making a prodrug is to select moieties which are hydrolyzed under physiological conditions to provide the desired compound. In other embodiments, the prodrug is converted by an enzymatic activity of the host animal or the target bacteria.

The term “treating” is art-recognized and refers to curing as well as ameliorating at least one symptom of any condition or disease.

The term “contacting” is art-recognized and refers to any method of delivering an adenosine A1 receptor (A1R) agonist, for example, but not limited to, N6-cyclopentyladenosine (CPA) and/or any other pharmaceutical, drug or therapeutic compound to hippocampal or other neurological tissue. In vitro or ex vivo this may be by exposing the hippocampal or other neurological tissue to the A1R agonist, pharmaceutical, etc. in a suitable medium, solution or bath. In vivo any known method of administration of the A1R agonist, pharmaceutical, etc. is suitable as described herein.

Thus, in one embodiment of the present invention, there is provided a method of controlling hippocampal neural circuit hyperexcitability occurring in a neurological disease or disorder associated with epileptogenesis in a subject in need of such treatment, comprising the step of contacting the hippocampus in said subject with a compound effective to restore excitatory/inhibitor balance thereby controlling the neural circuit hyperexcitability. Representative examples of useful compounds include but are not limited to adenosine, an adenosine mimetic, an adenosine modulator, an adenosine transport inhibitor and an adenosine receptor agonist. Representative examples of adenosine receptor agonists include but are not limited to a adenosine receptor congener, N6-cyclopentyladenosine, N6-cyclohexyladenosine, 2-chloro-cyclopentyladenosine, N-(3(R))-tetrahydrofuranyl)-6-aminopurine riboside, or a nucleoside transporter. Representative examples of adenosine transport inhibitors include but are not limited to a dipyridamole, nitrobenzylthioinosine, dilazep, benzodiazepine, dihydropyridies, xanthine or quinoline derivatives. Representative examples of adenosine modulators include but are not limited to an ecto-5′-nucleotidase inhibitor, an adenosine kinase inhibitor, a S-adenosylhomocysteine hydrolase inhibitor, and an adenosine diaminase inhibitor. Representative examples of a subject include but are not limited to one with intractable epilepsy, Dravet syndrome, febrile seizures, autism spectrum disorder or attention deficit hyperactivity disorder. This method may further comprise the step of administering a GABA modulating composition, an anticonvulsant agent, an ion channel inactivator, or a combination thereof. Representative examples of a GABA-modulating composition include but are not limited to barbiturates, benzodiazepines, Gabapentin, Pregabalin, 4-aminobutanoic acid (GABA), 4-amino-3-(4-chlorophenyl)butanoic acid (baclofen), 4-amino-3-phenylbutanoic acid, 4-amino-3-hydroxybutanoic acid, 4-amino-3-(4-chlorophenyl)-3-hydroxyphenylbutanoic acid, 4-amino-3-(thien-2-yl)butanoic acid, 4-amino-3-(5-chlorothien-2-yl)butanoic acid, 4-amino-3-(5-bromothien-2-yl)butanoic acid, 4-amino-3-(5-methylthien-2-yl)butanoic acid, 4-amino-3-(2-imidazolyl)butanoic acid, 4-guanidino-3-(4-chlorophenyl)butanoic acid, (3-aminopropyl)phosphonous acid, (4-aminobut-2-yl)phosphonous acid, sodium butyrate, (3-amino-2-methylpropyl)phosphonous acid, (3-aminobutyl)phosphonous acid, (3-amino-2-(4-chlorophenyl)propyl)phosphonous acid, (3-amino-2-(4-chlorophenyl)-2-hydroxypropyl)phosphonous acid, (3-amino-2-(4-fluorophenyl)propyl)phosphonous acid, (3-amino-2-phenylpropyl)phosphonous acid, (3-amino-2-hydroxypropyl)phosphonous acid, (E)-(3-aminopropen-1-yl)phosphonous acid, (3-amino-2-cyclohexylpropyl)phosphonous acid, (3-amino-2-benzylpropyl)phosphonous acid, [3-amino-2-(4-methylphenyl)propyl]phosphonous acid, [3-amino-2-(4-trifluoromethylphenyl)propyl]phosphonous acid, [3-amino-2-(4-methoxyphenyl)propyl]phosphonous acid, [3-amino-2-(4-chlorophenyl)-2-hydroxypropyl]phosphonous acid, (3-aminopropyl)methylphosphinic acid, (3-amino-2-hydroxypropyl)methylphosphinic acid, (3-aminopropyl)(difluoromethyl)phosphinic acid, (4-aminobut-2-yl)methylphosphinic acid, (3-amino-1-hydroxypropyl)methylphosphinic acid, (3-amino-2-hydroxypropyl)(difluoromethyl)phosphinic acid, (E)-(3-aminopropen-1-yl)methylphosphinic acid, (3-amino-2-oxo-propyl)methylphosphinic acid, (3-aminopropyl)hydroxymethylphosphinic acid, (5-aminopent-3-yemethylphosphinic acid, (4-amino-1,1,1-trifluorobut-2-yl)methylphosphinic acid, (3-amino-2-(4-chlorophenyl)propyl)sulfinic acid, and 3-aminopropylsulfinic acid.

In another embodiment of the present invention, there is provided a method of treating a neurological disease or disorder associated with epileptogenesis in a subject in need of such treatment, comprising the step of administering an amount of an adenosine A1 agonist pharmacologically effective to block epileptogenetic activities without blocking excitatory synaptic transmission. Representative examples of useful compounds include but are not limited to adenosine, an adenosine mimetic, an adenosine modulator, an adenosine transport inhibitor and an adenosine receptor agonist. Representative examples of adenosine receptor agonists include but are not limited to a adenosine receptor congener, N6-cyclopentyladenosine, N6-cyclohexyladenosine, 2-chloro-cyclopentyladenosine, N-(3(R))-tetrahydrofuranyl)-6-aminopurine riboside, or a nucleoside transporter. Representative examples of adenosine transport inhibitors include but are not limited to a dipyridamole, nitrobenzylthioinosine, dilazep, benzodiazepine, dihydropyridies, xanthine or quinoline derivatives. Representative examples of adenosine modulators include but are not limited to an ecto-5′-nucleotidase inhibitor, an adenosine kinase inhibitor, a S-adenosylhomocysteine hydrolase inhibitor, and an adenosine diaminase inhibitor. Representative examples of a neurological disease or disorder include but are not limited to an one with intractable epilepsy, Dravet syndrome, febrile seizures, autism spectrum disorder or attention deficit hyperactivity disorder. This method may further comprise the step of administering a GABA modulating composition, an anticonvulsant agent, an ion channel inactivator, or a combination thereof. Representative examples of a GABA-modulating composition include but are not limited to barbiturates, benzodiazepines, Gabapentin, Pregabalin, 4-aminobutanoic acid (GABA), 4-amino-3-(4-chlorophenyl)butanoic acid (baclofen), 4-amino-3-phenylbutanoic acid, 4-amino-3-hydroxybutanoic acid, 4-amino-3-(4-chlorophenyl)-3-hydroxyphenylbutanoic acid, 4-amino-3-(thien-2-yl)butanoic acid, 4-amino-3-(5-chlorothien-2-yl)butanoic acid, 4-amino-3-(5-bromothien-2-yl)butanoic acid, 4-amino-3-(5-methylthien-2-yl)butanoic acid, 4-amino-3-(2-imidazolyl)butanoic acid, 4-guanidino-3-(4-chlorophenyl)butanoic acid, (3-aminopropyl)phosphonous acid, (4-aminobut-2-yl)phosphonous acid, sodium butyrate, (3-amino-2-methylpropyl)phosphonous acid, (3-aminobutyl)phosphonous acid, (3-amino-2-(4-chlorophenyl)propyl)phosphonous acid, (3-amino-2-(4-chlorophenyl)-2-hydroxypropyl)phosphonous acid, (3-amino-2-(4-fluorophenyl)propyl)phosphonous acid, (3-amino-2-phenylpropyl)phosphonous acid, (3-amino-2-hydroxypropyl)phosphonous acid, (E)-(3-aminopropen-1-yl)phosphonous acid, (3-amino-2-cyclohexylpropyl)phosphonous acid, (3-amino-2-benzylpropyl)phosphonous acid, [3-amino-2-(4-methylphenyl)propyl]phosphonous acid, [3-amino-2-(4-trifluoromethylphenyl)propyl]phosphonous acid, [3-amino-2-(4-methoxyphenyl)propyl]phosphonous acid, [3-amino-2-(4-chlorophenyl)-2-hydroxypropyl]phosphonous acid, (3-aminopropyl)methylphosphinic acid, (3-amino-2-hydroxypropyl)methylphosphinic acid, (3-aminopropyl)(difluoromethyl)phosphinic acid, (4-aminobut-2-yl)methylphosphinic acid, (3-amino-1-hydroxypropyl)methylphosphinic acid, (3-amino-2-hydroxypropyl)(difluoromethyl)phosphinic acid, (E)-(3-aminopropen-1-yl)methylphosphinic acid, (3-amino-2-oxo-propyl)methylphosphinic acid, (3-aminopropyl)hydroxymethylphosphinic acid, (5-aminopent-3-yemethylphosphinic acid, (4-amino-1,1,1-trifluorobut-2-yl)methylphosphinic acid, (3-amino-2-(4-chlorophenyl)propyl)sulfinic acid, and 3-aminopropylsulfinic acid.

In yet another embodiment of the present invention, there is provided a method of treating severe myoclonic epilepsy in a subject in need of such treatment, comprising the step of administering an amount of an adenosine A1 agonist pharmacologically effective to treat said severe myoclonic epilepsy.

Representative examples of useful compounds include but are not limited to adenosine, an adenosine mimetic, an adenosine modulator, an adenosine transport inhibitor and an adenosine receptor agonist. Representative examples of adenosine receptor agonists include but are not limited to a adenosine receptor congener, N6-cyclopentyladenosine, N6-cyclohexyladenosine, 2-chloro-cyclopentyladenosine, N-(3(R))-tetrahydrofuranyl)-6-aminopurine riboside, or a nucleoside transporter. Representative examples of adenosine transport inhibitors include but are not limited to a dipyridamole, nitrobenzylthioinosine, dilazep, benzodiazepine, dihydropyridies, xanthine or quinoline derivatives. Representative examples of adenosine modulators include but are not limited to an ecto-5′-nucleotidase inhibitor, an adenosine kinase inhibitor, a S-adenosylhomocysteine hydrolase inhibitor, and an adenosine diaminase inhibitor. This method may further comprising the step of administering a GABA modulating composition, an anticonvulsant agent, an ion channel inactivator, or a combination thereof. Representative examples of a GABA-modulating composition include but are not limited to barbiturates, benzodiazepines, Gabapentin, Pregabalin, 4-aminobutanoic acid (GABA), 4-amino-3-(4-chlorophenyl)butanoic acid (baclofen), 4-amino-3-phenylbutanoic acid, 4-amino-3-hydroxybutanoic acid, 4-amino-3-(4-chlorophenyl)-3-hydroxyphenylbutanoic acid, 4-amino-3-(thien-2-yl)butanoic acid, 4-amino-3-(5-chlorothien-2-yl)butanoic acid, 4-amino-3-(5-bromothien-2-yl)butanoic acid, 4-amino-3-(5-methylthien-2-yl)butanoic acid, 4-amino-3-(2-imidazolyl)butanoic acid, 4-guanidino-3-(4-chlorophenyl)butanoic acid, (3-aminopropyl)phosphonous acid, (4-aminobut-2-yl)phosphonous acid, sodium butyrate, (3-amino-2-methylpropyl)phosphonous acid, (3-aminobutyl)phosphonous acid, (3-amino-2-(4-chlorophenyl)propyl)phosphonous acid, (3-amino-2-(4-chlorophenyl)-2-hydroxypropyl)phosphonous acid, (3-amino-2-(4-fluorophenyl)propyl)phosphonous acid, (3-amino-2-phenylpropyl)phosphonous acid, (3-amino-2-hydroxypropyl)phosphonous acid, (E)-(3-aminopropen-1-yl)phosphonous acid, (3-amino-2-cyclohexylpropyl)phosphonous acid, (3-amino-2-benzylpropyl)phosphonous acid, [3-amino-2-(4-methylphenyl)propyl]phosphonous acid, [3-amino-2-(4-trifluoromethylphenyl)propyl]phosphonous acid, [3-amino-2-(4-methoxyphenyl)propyl]phosphonous acid, [3-amino-2-(4-chlorophenyl)-2-hydroxypropyl]phosphonous acid, (3-aminopropyl)methylphosphinic acid, (3-amino-2-hydroxypropyl)methylphosphinic acid, (3-aminopropyl)(difluoromethyl)phosphinic acid, (4-aminobut-2-yl)methylphosphinic acid, (3-amino-1-hydroxypropyl)methylphosphinic acid, (3-amino-2-hydroxypropyl)(difluoromethyl)phosphinic acid, (E)-(3-aminopropen-1-yl)methylphosphinic acid, (3-amino-2-oxo-propyl)methylphosphinic acid, (3-aminopropyl)hydroxymethylphosphinic acid, (5-aminopent-3-yemethylphosphinic acid, (4-amino-1,1,1-trifluorobut-2-yl)methylphosphinic acid, (3-amino-2-(4-chlorophenyl)propyl)sulfinic acid, and 3-aminopropylsulfinic acid.

The dosage of any compositions of the present invention will vary depending on the symptoms, age and body weight of the patient, the nature and severity of the disorder to be treated or prevented, the route of administration, and the form of the subject composition. Any of the subject formulations may be administered in a single dose or in divided doses. Dosages for the compositions of the present invention may be readily determined by techniques known to those of skill in the art or as taught herein.

In certain embodiments, the dosage of the subject compounds will generally be in the range of about 0.01 ng to about 10 g per kg body weight, specifically in the range of about 1 ng to about 0.1 g per kg, and more specifically in the range of about 100 ng to about 10 mg per kg.

An effective dose or amount, and any possible affects on the timing of administration of the formulation, may need to be identified for any particular composition of the present invention. This may be accomplished by routine experiment as described herein, using one or more groups of animals (preferably at least 5 animals per group), or in human trials if appropriate. The effectiveness of any subject composition and method of treatment or prevention may be assessed by administering the composition and assessing the effect of the administration by measuring one or more applicable indices, and comparing the post-treatment values of these indices to the values of the same indices prior to treatment.

The precise time of administration and amount of any particular subject composition that will yield the most effective treatment in a given patient will depend upon the activity, pharmacokinetics, and bioavailability of a subject composition, physiological condition of the patient (including age, sex, disease type and stage, general physical condition, responsiveness to a given dosage and type of medication), route of administration, and the like. The guidelines presented herein may be used to optimize the treatment, e.g., determining the optimal time and/or amount of administration, which will require no more than routine experimentation consisting of monitoring the subject and adjusting the dosage and/or timing.

While the subject is being treated, the health of the patient may be monitored by measuring one or more of the relevant indices at predetermined times during the treatment period. Treatment, including composition, amounts, times of administration and formulation, may be optimized according to the results of such monitoring. The patient may be periodically reevaluated to determine the extent of improvement by measuring the same parameters. Adjustments to the amount(s) of subject composition administered and possibly to the time of administration may be made based on these reevaluations. Treatment may be initiated with smaller dosages which are less than the optimal dose of the compound. The dosage may be increased by small increments until the optimal therapeutic effect is attained.

Agents of the present invention can be administered orally, parenterally, for example, subcutaneously, intravenously, intramuscularly, intraperitoneally, by intranasal instillation, or by application to mucous membranes, such as, that of the nose, throat, and bronchial tubes. They may be administered alone or with suitable pharmaceutical carriers, and can be in solid or liquid form such as, tablets, capsules, powders, solutions, suspensions, or emulsions.

The active agents of the present invention may be orally administered, for example, with an inert diluent, or with an assimilable edible carrier, or they may be enclosed in hard or soft shell capsules, or they may be compressed into tablets, or they may be incorporated directly with the food of the diet. For oral therapeutic administration, these active agents may be incorporated with excipients and used in the form of tablets, capsules, elixirs, suspensions, syrups, and the like. Such compositions and preparations should contain at least 0.1% of active agent. The percentage of the agent in these compositions may, of course, be varied and may conveniently be between about 2% to about 60% of the weight of the unit. The amount of active agent in such therapeutically useful compositions is such that a suitable dosage will be obtained. Preferred compositions according to the present invention are prepared so that an oral dosage unit contains between about 1 and 250 mg of active agent.

The tablets, capsules, and the like may also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, or saccharin. When the dosage unit form is a capsule, it may contain, in addition to materials of the above type, a liquid carrier, such as a fatty oil. Various other materials may be present as coatings or to modify the physical form of the dosage unit. For instance, tablets may be coated with shellac, sugar, or both. A syrup may contain, in addition to the active ingredient, sucrose as a sweetening agent, methyl and propylparabens as preservatives, a dye, and flavoring such as cherry or orange flavor.

These active agents may also be administered parenterally. Solutions or suspensions of these active agents can be prepared in water suitably mixed with a surfactant, such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols such as, propylene glycol or polyethylene glycol, are preferred liquid carriers, particularly for injectable solutions. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

The agents of the present invention may also be administered directly to the airways in the form of an aerosol. For use as aerosols, the agents of the present invention in solution or suspension may be packaged in a pressurized aerosol container together with suitable propellants, for example, hydrocarbon propellants like propane, butane, or isobutane with conventional adjuvants. The materials of the present invention also may be administered in a non-pressurized form such as in a nebulizer or atomizer.

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

### Example 1

### Animals

The model of SMEI used in these studies is caused by a knock-in nonsense substitution (CgG to TgA in exon 21) made within a loop between segments 5 and 6. Transgenic mice were provided by Drs. K. Yamakawa and I. Ogiwara (RIKEN, Japan (14)). All of the experiments on the mice (C57BL/6/129) involved in this project were performed in accordance with animal protocols approved by the Institutional Animal Care and Use Committee of the University of Houston. Heterozygous (HET) and wild-type (WT) mice were used.

### Example 2

### Slice Preparation

Mice (P16-22) were anesthetized with isofluorane, decapitated, and the brains immediately removed. Transverse hippocampal sections (350 μm thickness) were cut in cold dissection solution (in mM: 2.6 KCl, 1.23 NaH₂PO₄, 24 NaHCO₃, 0.1 CaCl₂, 2 MgCl₂, 205 sucrose, and 10 glucose) using a vibratome and were incubated for half an hour in normal artificial cerebrospinal fluid (ACSF; pH 7.3, 30° C.) containing (in mM) 130 NaCl, 1.2 MgSO₄, 3.5 KCl, 1.2 CaCl₂, 10 glucose, 2.5 NaH₂PO₄, 24 NaHCO₃ aerated with 95% O₂-5% CO₂. After the incubation, slices were stained with Di-4ANNEPS (final dye concentration is 0.05 mg/ml) and left to recover for an additional hour at 30° C. (23). The slices were transferred to a submersion recording chamber (Warner Instr.) and continuously perfused (2 ml/min, at 30° C.) with the oxygenated ACSF.

### Example 3

### Optical Imaging and Electrical Recordings

A combination of in vitro electrophysiology (extracellular field potential recording and whole-cell patch clamp recordings) and fast voltage-sensitive dye imaging (VSDI) was used. All electrical recordings were performed using MCC 700 amplifiers (Axon Instruments). Electrical data was acquired at 4 KHz, digitized at 10 KHz using a Digidata DAC board and pClamp software. Optical data were recorded at 250 Hz with MiCam 02 (192×126 pixels, SciMedia USA).

For electrical whole-cell voltage-clamp recordings, borosilicate glass micropipettes (4-7 MΩ) were used containing (in mM): 116 Cesium gluconate, 6 KCl, 0.5 EGTA, 20 HEPES, 10 phosphocreatine, 0.3 NaGTP, 2 NaCl, 4 MgATP, and 0.3% Neurobiotin (pH 7.25, 295 mOsm) and 5 mM QX-314 (fast voltage-gated conductance blocker). Spontaneous inhibitory postsynaptic currents (IPSCs) and EPSCs were recorded in CA1 pyramidal cells. sIPSCs were recorded at −80 mV in the presence of APV (100 μM) and CNQX (40 μM). sEPSCs were recorded at −70 mV in the presence of inhibitory synaptic transmission blocker picrotoxin (50 μM). Amplitude and frequency of sEPSCs and sIPSCs were measured and compared between WT and HET. For IPSC calculations, activity was studied in three 20 second segments (twenty seconds apart) for each cell (N=8 HET, 6WT). Since EPSCs have lower frequency, two minute long segments were used for their analysis (N=6). Cumulative distribution of the amplitudes and inter-event interval (frequency) were plotted in Prism software. t-test analysis was used to compare IPSCs and EPSCs in HET and WT tissue. All recordings were performed at 32° C. temperature.

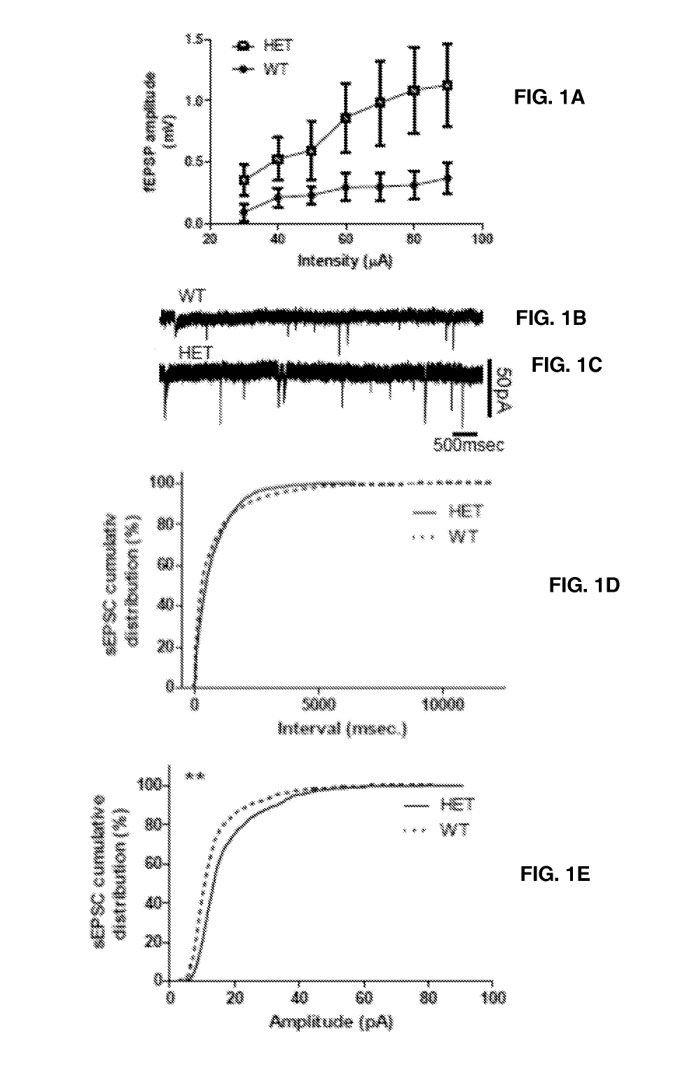

To characterize excitatory neural circuit activity, stimulating electrodes (concentric bipolar metal electrodes, 200 μm in diameter (FHC)) were placed on the Shaffer collaterals. fEPSP recordings were performed in hippocampal slices concurrently with the VSDI (FIGS. 1A-1E). Extracellular recording electrodes (1-2 mΩ, 0.9% saline) were placed in the CA1 radiatum layer. To monitor Di-4ANNEPS signals, the slices were illuminated either with Halogen (150 W; excitation 522-550 nm; emission—580 nm; dichroic—565 nm).

Input-output (I-O) characteristics of fEPSPs were calculated using the same intensities of stimulation and incrementally (0.05 mA) raising them until the maximal responses or population spikes were obtained. 1-0 calculations were performed in stained and unstained slices to rule out a possible modulation of Di-4ANEPPS on GABA receptors (24). The responses from stained and unstained slices were not statistically different and were pooled together for the final analysis.

To elicit short-term synaptic plasticity, Schaeffer collaterals were stimulated at 40 Hz frequency using 10 pulse (0.2 msec) trains. 40 Hz falls within the gamma frequency range and stimulation was repeated every 15 seconds with stimulus amplitude was set at the half of maximal fEPSP amplitude. fEPSP and VSDI optical data trials were synchronized.

To modulate hippocampal hyperexcitability A1R agonist N6-cyclopentyladenosine (CPA) was used. N6-cyclopentyladenosine dissolved in DMSO was added to the bath ACSF solution. N6-cyclopentyladenosine took effect within 2 minutes of its application. 10 and 1 μM and 500, 100, 50, and 10 nM concentrations of CPA were tested. Since N6-cyclopentyladenosine acts through G-protein linked mechanisms, the washout of N6-cyclopentyladenosine was not possible in the current experiments. In a subset of control experiments (n=5 slices), we confirmed that the vehicle DMSO on its own does not affect the size of fEPSP responses. A1R agonist DPCPX concentration was 200 nM. All drugs and dye were obtained from Sigma-Aldrich.

### Example 4

### Data and Statistical Analysis

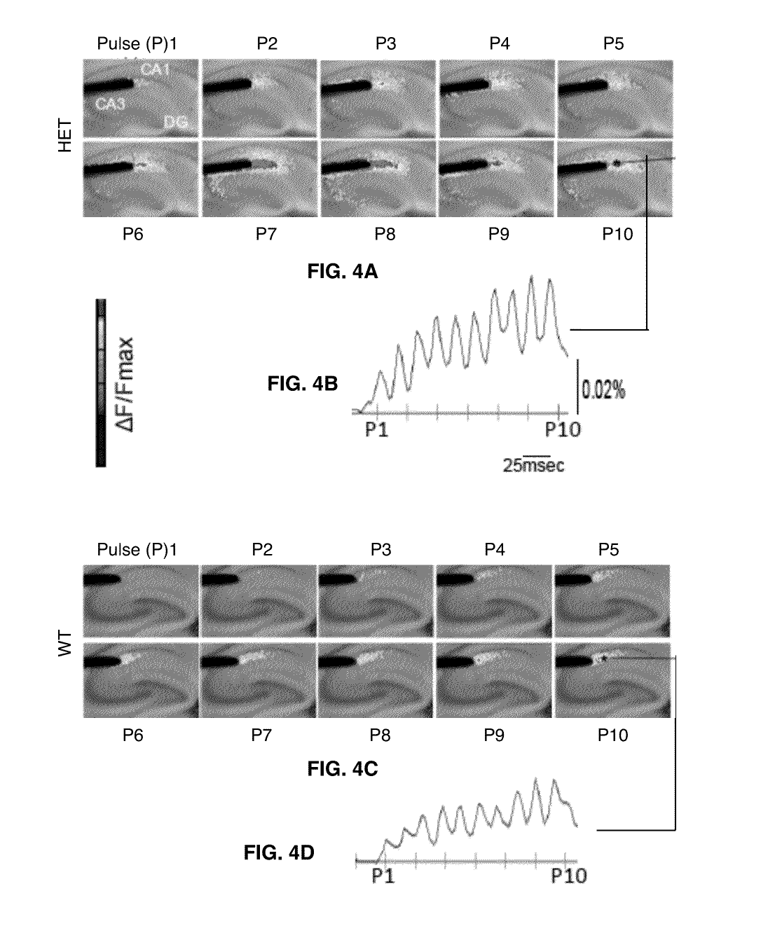

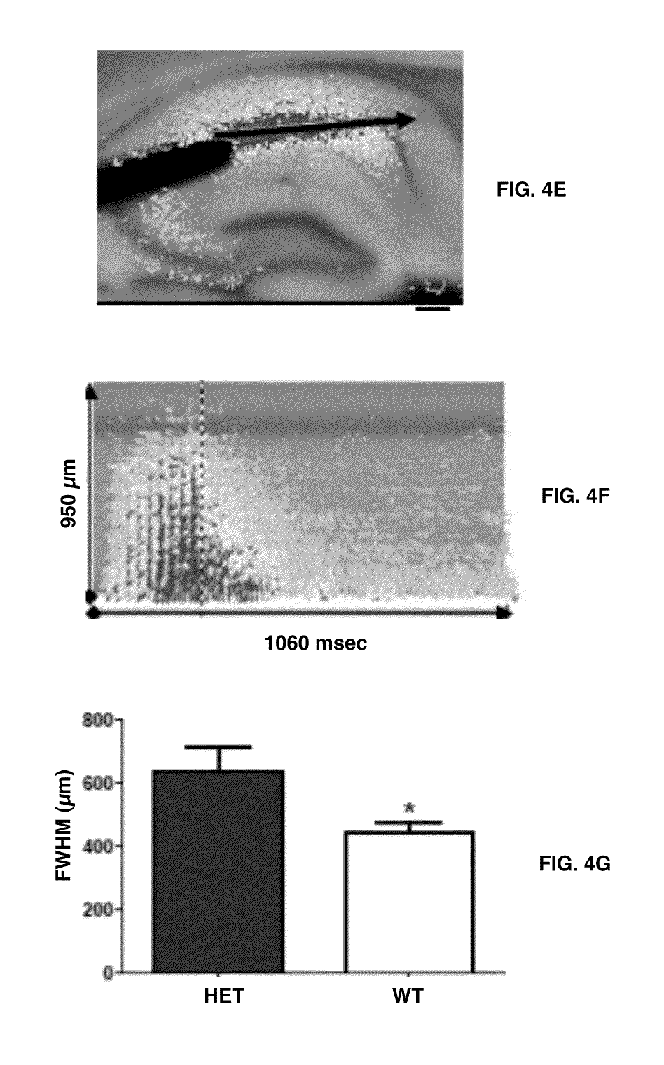

Optical and electrical data were analyzed using Brain Vision and pClamp softwares. To increase signal to noise ratio, data analysis in individual slices and during pharmacological manipulations was performed on the averages of fifteen files (electrical and optical). Standard electrophysiological analysis techniques were used to analyze fEPSP, EPSC, and IPSC characteristics. For optical analysis, 1060 msec of data were fitted with an approximately Gaussian curve (25) and full width at half maximal (FWHM) values over the distance of 950 micrometers from the stimulating electrode toward subicullum were calculated using BrainVision (SciMedia). FWHM here quantifies distance of neuronal signal propagation (or decay). FWHM calculations along the orthodromic neural activity propagation trajectory included 15 frames before and 194 frames after the 40 Hz train stimulation, and was spanning over the evoked signal as shown in FIGS. 4A-4D. All results are reported as grouped averages with standard error of the mean. Results from WT and HET, or treated versus untreated groups were compared using unpaired and paired t-tests, respectfully. A p<0.05 was regarded as statistically significant value.

### Example 5

### Increased Synaptic Excitation in the SCN1A mSMEI

To study the impact of SCN1A mutation on the synaptic excitation/inhibition balance in the hippocampus, extracellular and whole-cell patch clamp recordings were performed in the CA1 area and in the pyramidal cells, respectively. fEPSPs were evoked by Schaeffer collateral projection stimulation in the normal ACSF solution (FIGS. 1A-1E). fEPSP amplitudes were measured in wild-type (WT) and heterozygous (HET) transgenic tissue using the same stimulation intensities. Stimulation-response (or input-output) measurements showed that lower amplitude electrical stimulation is required to evoke larger amplitude fEPSPs in the HET mouse versus WT tissue (FIG. 1A). This suggested that synaptic excitation in SCN1A mutants is increased.

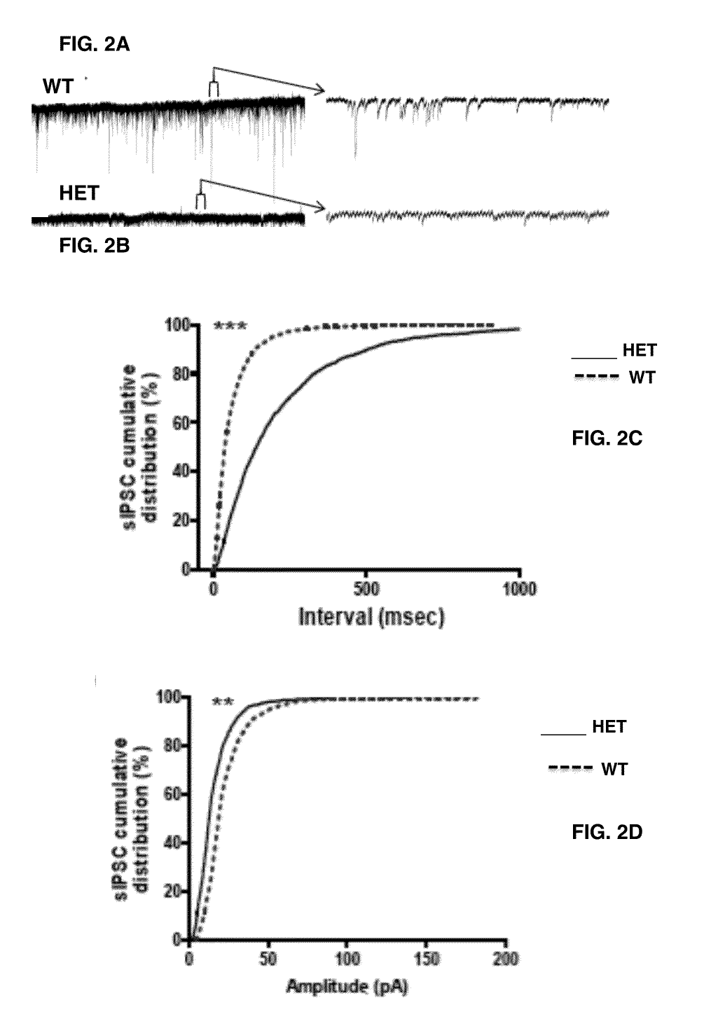

To further elucidate how the SCN1A mutation affects synaptic excitation, whole-cell patch clamp recordings of spontaneous excitatory postsynaptic currents (sEPSCs) were performed in the hippocampal CA1 excitatory pyramidal cells (FIGS. 2A-2B). sEPSC recordings were performed in the ACSF which contained inhibitory neurotransmission blocker picrotoxin (PTX, 50 μM). Recordings were done in the voltage-clamp mode at −70 mV using recording solution which contained cesium gluconate and QX-314. This allowed isolatation of synaptic sEPSCs and calculate cumulative distributions of the frequency of occurrence and amplitude (FIGS. 1D-1E). sEPSC frequency was not significantly different in HET from the WT tissues (FIG. 1D). However, sEPSC amplitudes were larger in the HET hippocampi (FIG. 1E). Thus, both the evoked and spontaneous excitatory responses are increased in the CA1 hippocampi of animals with SCN1A mutation.

### Example 6

### Impaired Synaptic Inhibition in mSMEI

To further elucidate the synaptic E/I balance, spontaneous inhibitory postsynaptic currents (sIPSCs) were examined (FIGS. 2A-2D). sIPSCs were recorded in the presence of glutamatergic transmission blockers (CNQX, 40 uM and APV, 100 uM) at −80 mV. Cumulative distributions of sIPSC frequency of occurrence and amplitude were calculated and statistically compared (FIGS. 2C-2D). sIPSCs were significantly less frequent and smaller in amplitude in the CA1 pyramidal cells from the HET tissue. Changes in the membrane input resistances could not account for the differences in sEPSC/sIPSC amplitudes. For sEPSC and sIPSC experiments, input resistance in the pyramidal CA1 cells in WT and HET animal tissue were statistically insignificant (WT: 190.4±11.49MΩ, N=14 cells and HET: 183.6±16.78 MΩ, N=11 cells).

### Example 7

### Impaired Synaptic Short-Term Plasticity (STP) in mSMEI

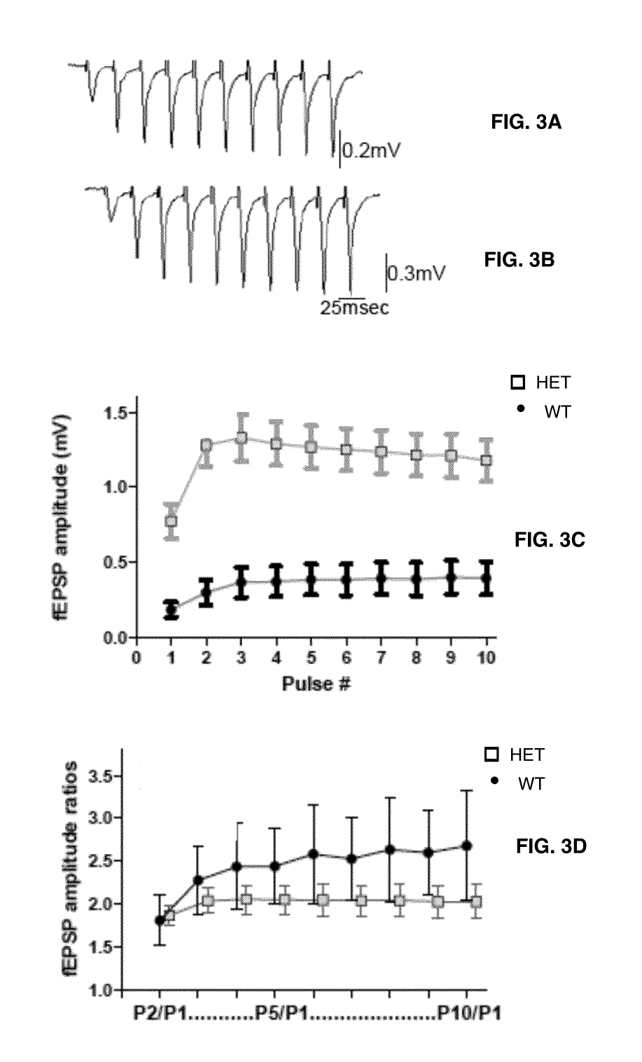

Significant E/I imbalance in the CA1 circuit led to further examination of synaptic activity in the CA1 synapses. STP and the spatial extent of neural signal propagation were examined during 40 Hz train stimulations. Electrical fEPSP recordings (FIGS. 3A-3D) were performed concurrently with the fast optical VSDI (FIGS. 4A-4G). Trains of ten fEPSPs were evoked and electrical measurements showed that fEPSP responses were significantly increased and sustained during the stimulation in the mSMEI tissue (FIG. 3A-3C). To quantify changes in the synaptic plasticity, amplitudes of fEPSPs within the train were compared (FIG. 3D). fEPSP amplitude ratios showed a significant divergence between responses in WT and HET tissue at the later parts of the evoked stimulation trains. At 40 Hz, CA1 synapses typically show facilitatory responses (26-29). Synapses in the WT tissue showed continuous facilitation throughout the stimulation train (FIG. 3D), which was significantly reduced in HET tissue, especially following the first two pulses. Taken together, increased excitability and impaired STP are potentially both contributing to epileptogenesis and impaired information processing within the hippocampal circuit.

### Example 8

### Aberrant Neural Activity Propagation in mSMEI

To further analyze CA1 circuit activity, the entire hippocampal circuit in the field of view was visualized using VSDI and determined if the E/I imbalance results in the increased spatial activation of the CA1 region. Fast optical acquisition (250 Hz) allowed capture of the evoked trains of activity using VSDI. When Schaeffer collaterals were stimulated, equivalent intensity of stimulation led to abnormally widespread CA1 neural activation maps in the HET tissue (FIGS. 4A-4D). This increased optical activation was in agreement with the increased fEPSP responses recorded in the HET tissue (FIG. 3B). In HET tissue, equivalent amplitudes of electrical stimulation as in HET activated larger areas or neural activity (FIGS. 4A-4D).

The difference in the propagation distance of the evoked bursts from the site of the stimulating electrode was calculated using full width at half maximal (FWHM) value of the peak normalized fluorescence measure (FIGS. 4E-4G; (25)). Evoked neural activity in the HET tissue was propagating significantly further distances, consistent with the E/I imbalance. It is also important to note that the 40 Hz trains often produced antidromic activation in the HET hippocampi (Pulses 6 and 7, FIG. 4A). Coupled with E/I imbalance this further suggests a significant disruption in the proper CA1 neural circuit activation pattern.

### Example 9

Adenosine A1 Receptor Agonist Modulation of CA1 Circuit Excitability

Adenosine, the core of ATP, has gained great interest recently as an endogenous anti-convulsant (30-31). The majority of its neuroprotective and anti-epileptic effects are mediated by the adenosine A1 receptor (A1R), which is ubiquitously expressed in the excitatory neurons. A1R acts through pre-synaptic G-protein coupled receptors, reducing calcium influx into synaptic terminals, increasing potassium currents, and inhibiting the release of glutamate (32). A1R agonist, however is a novel approach to treat Dravet syndrome. Thus, to determine the effects of adenosine agonist on normal and transgenic neural circuit activity, the A1R agonist N6-cyclopentyladenosine was used.

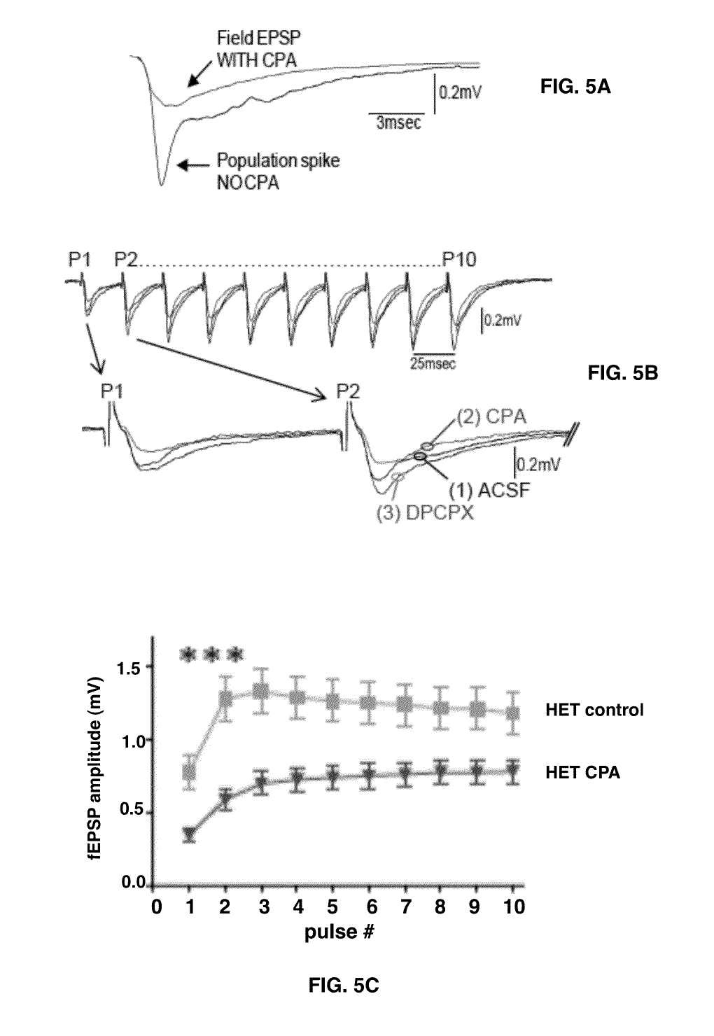

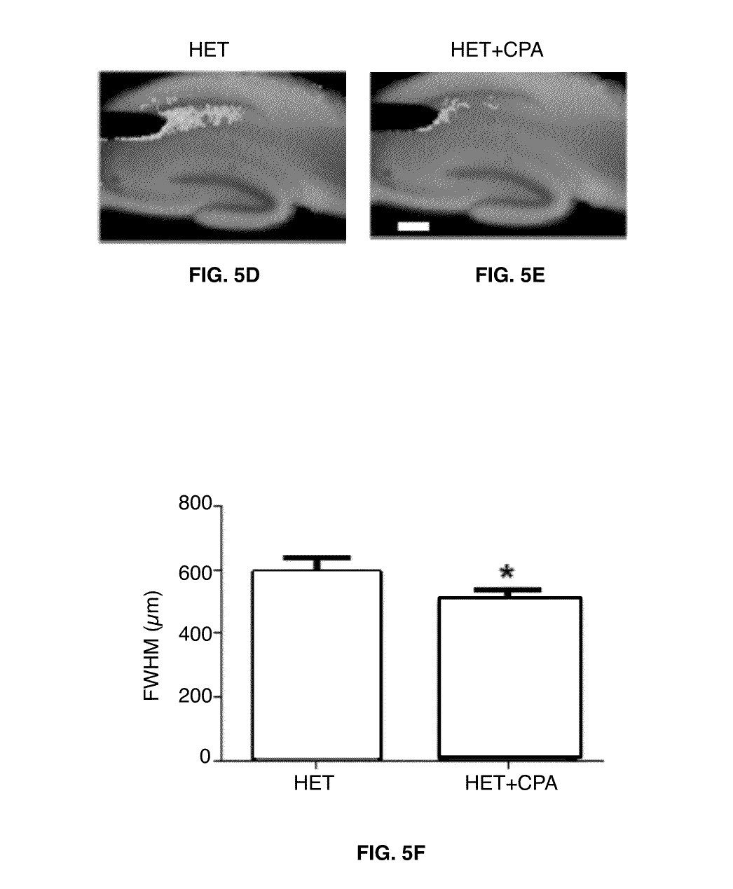

Initial experiments using 1 and 10 μM concentrations of N6-cyclopentyladenosine (33-34) showed that at these concentrations N6-cyclopentyladenosine completely eliminated fEPSPs. Using N6-cyclopentyladenosine concentrations of 500, 100, 50, and 10 nM, it was found that the 50 nM concentration was sufficient at significantly reducing synaptic excitability in HET tissues, without blocking synaptic transmission (FIGS. 5A-5D). Data presented here is obtained using 50 nM concentration of N6-cyclopentyladenosine. In some instances, where low stimulation amplitude would result in the population spikes responses in the CA1 of HET tissue, 50 nM N6-cyclopentyladenosine effectively reduced this exaggerated response into fEPSP (FIG. 5A). To confirm that N6-cyclopentyladenosine is acting through the adenosine receptor, we used adenosine receptor antagonist DPCPX (FIG. 5B). N6-cyclopentyladenosine reliably reduced the evoked responses in the control ACSF. However, when DPCPX (200 nM) was added to the solution that contained the A1R agonist, N6-cyclopentyladenosine was ineffective at reducing fEPSP.

To determine if N6-cyclopentyladenosine was also effective at reducing the spread of neural activity during 40 Hz train stimulations, the optical signals were analyzed. VSDI measurements and calculations showed that N6-cyclopentyladenosine reduced propagation of the evoked signal from the stimulation site into area CA1 (FIGS. 5B-5F). This suggests that A1R agonists may be of interest as an alternative for controlling spread of the aberrant epileptic activity in SMEI circuits.

### Example 10

### Synaptic Impairments Caused by SCN1A Mutation

Normal neural function requires finely tuned and balanced excitation and inhibition (35-36). For this balance to exist, inhibitory and excitatory neurons must reliably generate action potentials using voltage-gated sodium and potassium channels (37-38). However, mutations that affect the SCN1A gene and impair functions of Naᵥ1.1 channel proteins result in decreased sodium-mediated action potential firing in the specific subset of parvalbumin-positive inhibitory interneurons (14). This impairment is specific to neocortical and hippocampal interneurons and has not been reported to affect pyramidal cell action potential generation or levels of excitation. Surprisingly, most of the in vitro experiments on SCN1A mutation have been performed using model cell cultures and isolated cells. Slice physiology studies in this mutant are limited and synaptic and circuit level alterations that precede the initial seizures are poorly understood.

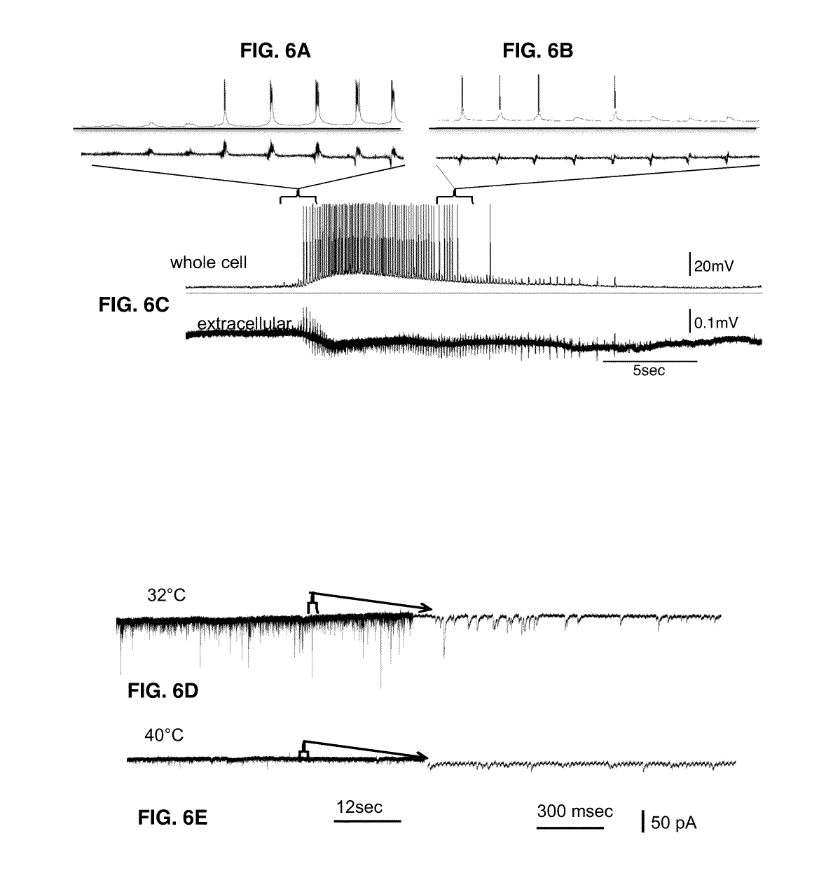

E/I imbalance in the SCN1A mutant during the third postnatal week are due to both, loss of synaptic inhibition and increased excitation. Loss of inhibition is consistent with the Nav1.1 location in the inhibitory cells. Increase in the spontaneous and evoked excitation levels in mSMEI tissue suggest an additional, compounding problem. Increased initial fEPSP responses (FIGS. 6A-6E) indicate that the CA1 excitatory synapses in HET tissue are potentiated. Furthermore, the results with STP measurements indicate that the hippocampal CA1 synapses are not as malleable. Inability of the synapses to properly respond to the incoming stimuli may result in the improper activation of the circuitry and abnormal information processing, as well as serve as an alternative mechanism for epileptogenesis. On the other hand, decreased facilitation observed in HET tissue could also be a compensatory mechanism that prevents even further facilitation in these already hyperexcitable synapses. The severe E/I imbalance caused by the SCN1A mutation can affect some of the key neuronal circuits and serve as a prelude for early life febrile seizures and the associated sequel of cognitive and social dysfunctions.

### Example 11

### Modulation of Hyperthermia-Induced Seizures

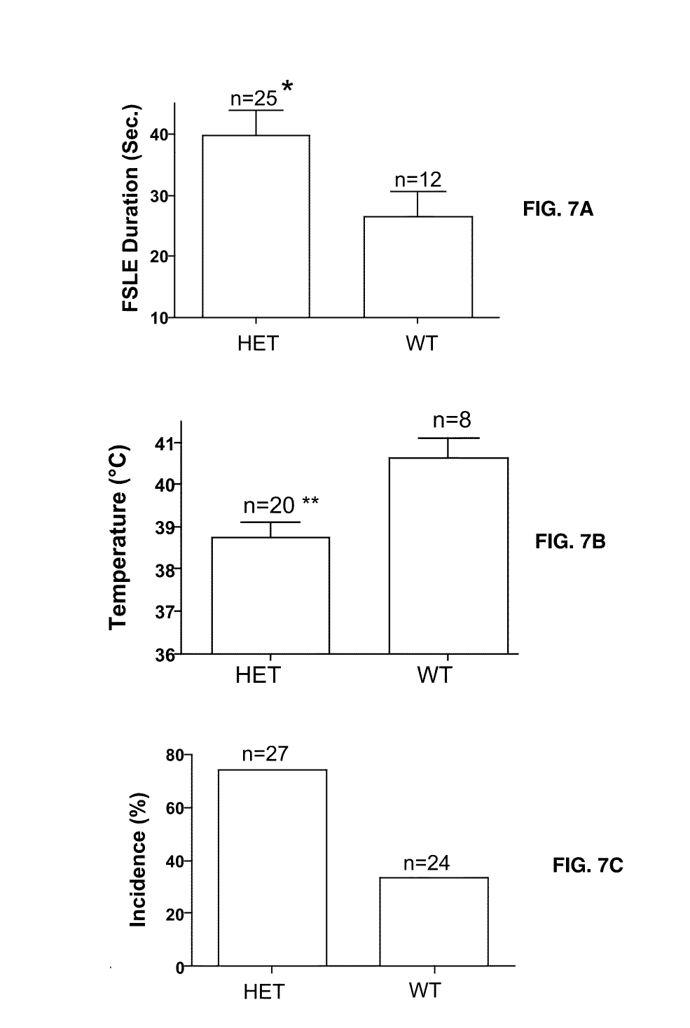

Febrile seizure-like events (FSLE) emerge and terminate as sub-threshold bursts under conditions of hyperthermia at a temperature of about 39° C. in (FIGS. 6A-6C) while spontaneous inhibitory postsynaptic currents (IPSCs) evident at 32° C. (FIG. 6D) gradually diminish as the temperature increases (FIG. 6E). In hippocampal slices FSLEs emerged on the average at 38.5° C. in tissue from HET mice and at 40.5° C. in tissue from WT mice (FIGS. 7A-7B) where the FSLE durations was almost twice as long in HET tissue (FIG. 7C).

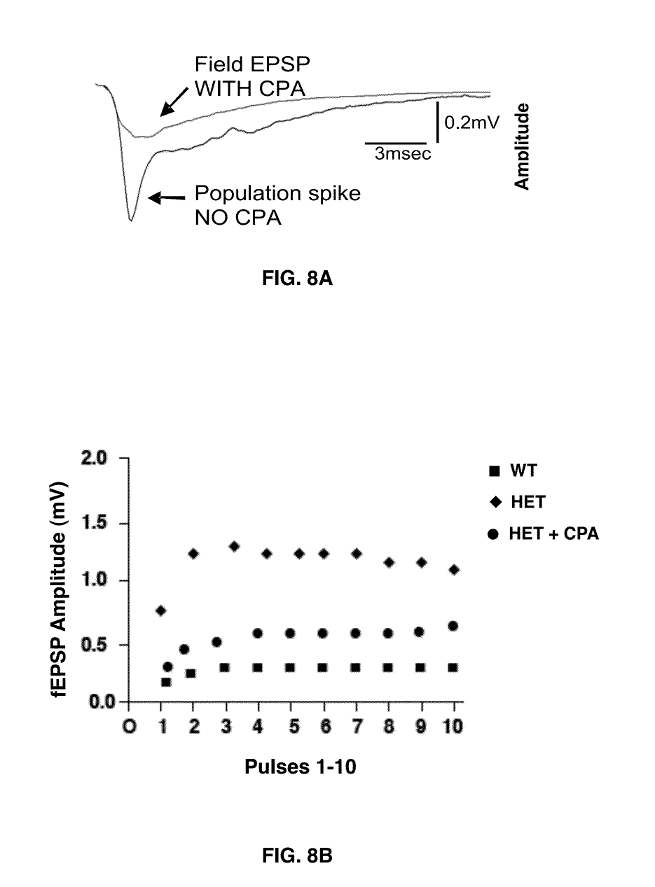

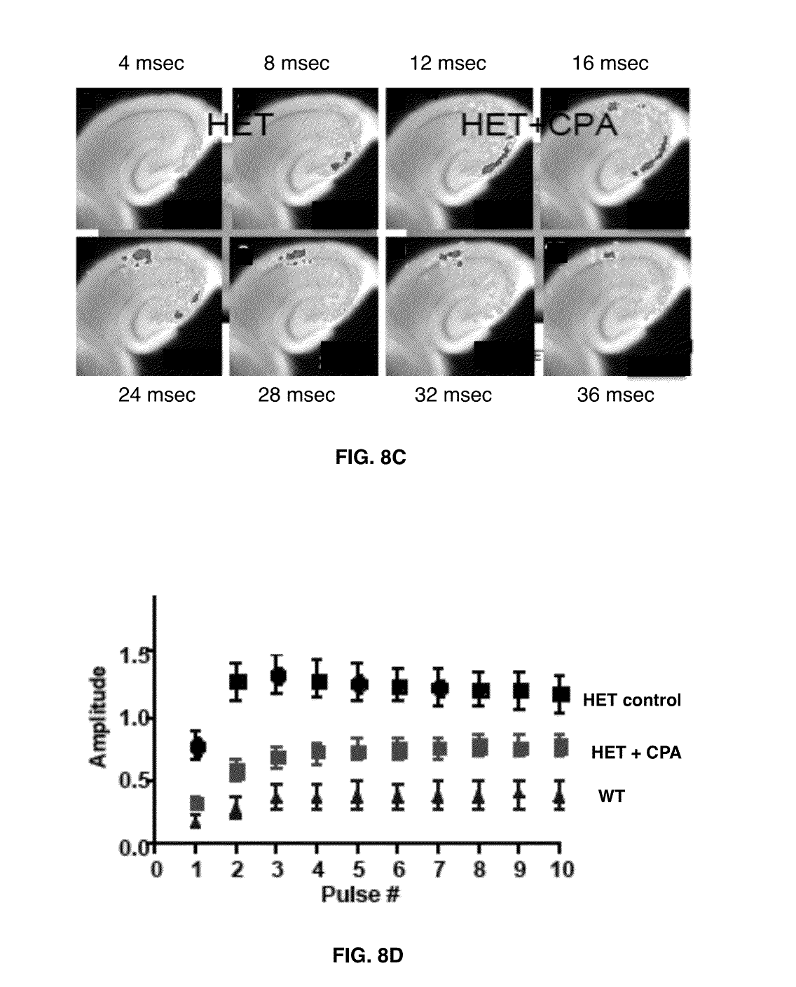

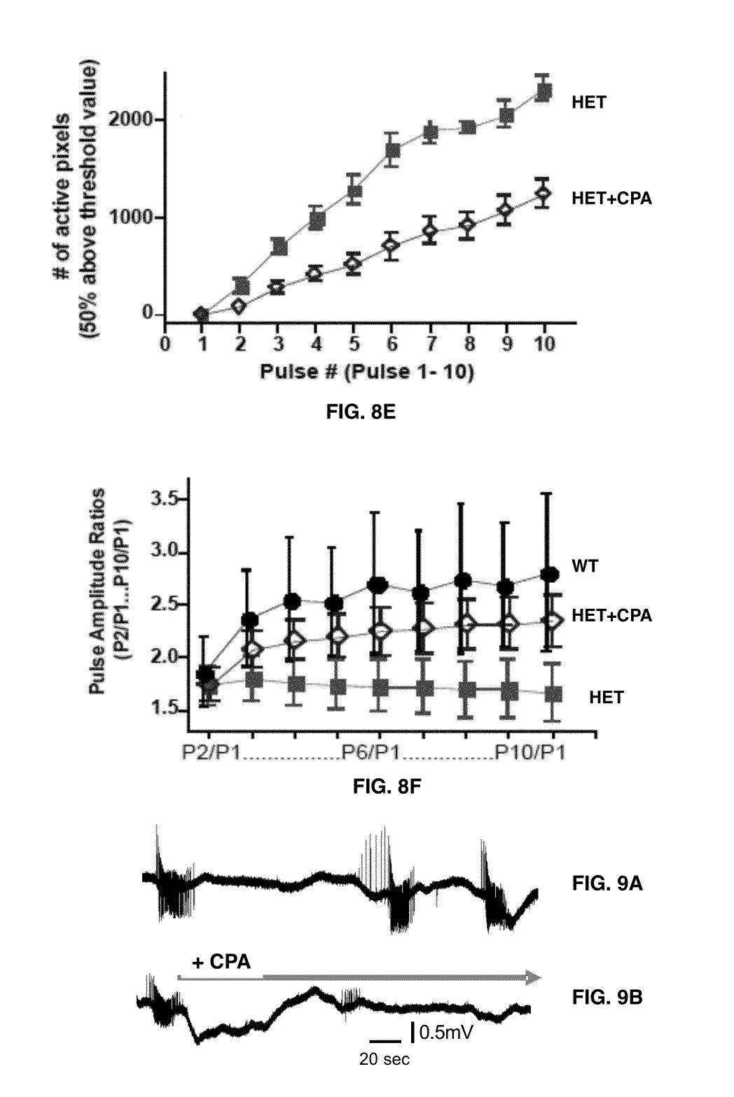

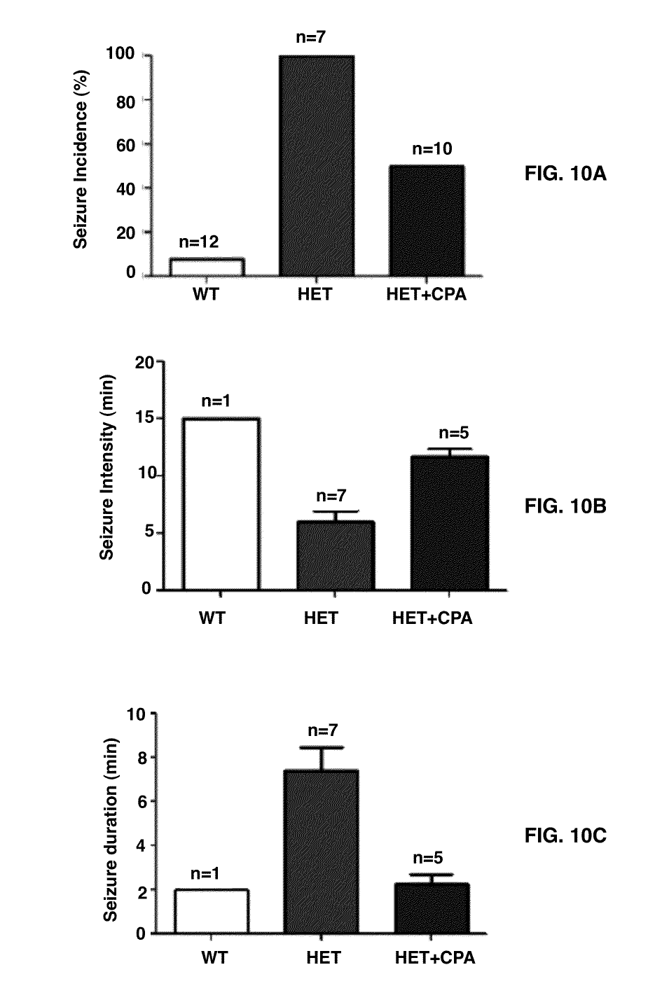

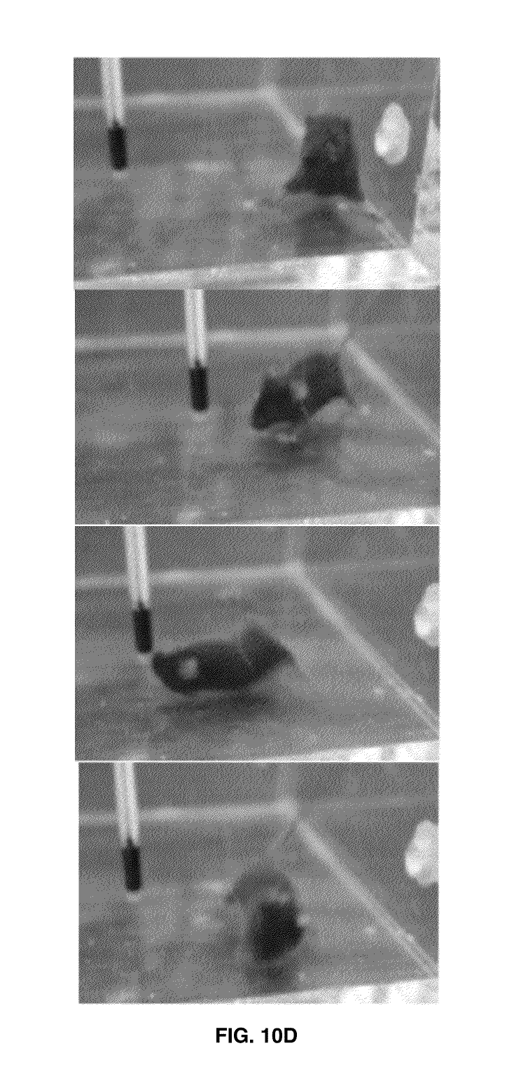

After contact with CPA, population spikes were reduced into a field EPSP response (FIG. 8A). Average fEPSPs (FIG. 8B) and the abnormally wide circuit excitation (FIGS. 8C-8D) were reduced significantly in the HET hippocampus. Moreover, CPA significantly reduced the spatial extent of neural signal propagation (FIG. 8E) and effected the short-term plasticity in HET+CPA mice (FIG. 8F). It was demonstrated that, in isolated mouse hippocampus, CPA blocked epileptogenic activity in hyperthermia (FIGS. 9A-9B). Mice with SCN1A mutation have a lower febrile seizure threshold (FIGS. 10A, 10D). After hyperthermia, febrile seizures occurred at shorter latency (FIG. 10B) and were of longer duration (FIG. 10C) than in a WT mouse having a seizure.

### Example 12

### Fast Functional Imaging of E/I Imbalance in Neural Circuits

Microcircuit and larger scale imaging modalities provide important tools for understanding the interactions between various, heterogeneous brain regions and the cells within them. VSDI provides a way to simultaneously measure the membrane potential of neurons across wide spatial areas and to identify regions that drive epileptiform activity. VSDI signals are linearly correlated with post synaptic neuronal membrane potential fluctuations (18). In chronic epilepsy models, evoked neuronal signals imaged using VSDI all show a substantially wider area of activation compared with the area activated in their wild-type counterparts (39). In fact, some of the fundamental knowledge about neuronal ‘wave’ propagation came from the studies which utilized evoked activity in the presence of inhibitory synaptic blockers (40), arguably mimicking epileptogenic-like state. Previous work in the 4-aminopyridine model using VSDI showed that increases in synchrony even during shorter duration interictal bursts are also associated with the wider area of burst propagation in the hippocampus (23).

At present, synaptic activity studied using a combination of electrophysiology and VSDI allowed visualizing neural circuit activity in the transgenic model of pediatric epilepsy for the first time. The present invention shows that the previously reported loss of inhibition results in the CA1 circuit-wide dysfunction is reflected by abnormally wide area of the evoked excitatory signal propagation in the transgenic tissue. Evoked responses in HET tissue were also more likely to exhibit antidromic activation, suggesting that loss of functional inhibition would make SMEI circuit activation anatomically non-discriminating. Furthermore, these results suggest that the reported loss of inhibition from the subset of the parvalbumin-positive inhibitory cells expressing Nav1.1 is not compensated by the other inhibitory CA1 neuron subpopulations. Spontaneous IPSCs on the pyramidal cells were decreased and these IPSCs are likely comprised of the diverse subset of perisomatically projecting inhibitory neurons. Thus, one may elucidate if the other pathways connecting hippocampal and entorhinal cortices are also affected by the SCN1A mutation, and to determine how individual excitatory and inhibitory cell activity (41) dynamically creates epileptogenic zones in SMEI. Conceivably, increased hyperexcitability in the CA1 could be compensated by the surrounding hippocampal regions, for example, via decreased excitation or increased inhibition along the perforant or the mossy fiber pathways of the hippocampus.

### Example 13

### Modulating Network Hyperexcitability with A1R Agonist

SMEI remains one of the most pharmacoresistant forms of epilepsy. Currently, to treat SMEI seizures GABA modulators are used to enhance the inhibition. Valproate is commonly used to prevent the recurrence of febrile seizures, and benzodiazepines are used for long-lasting seizures, but they are often insufficient (16). Some other drugs, like lamotrigine, carbamazepine, phenobarbital were also previously tested, but none of these agents worked reliably (16, 42). Increasing GABA synthesis may work well in the networks that contain functionally intact inhibitory cells. Unfortunately, in many forms of epilepsy including the SMEI models, the inhibitory neurons are affected and may lose their ability to fire action potentials and potentially release GABA. Furthermore, in hyperexcitable networks and during epileptic activity, excitatory cells often get loaded with chloride, which results in the GABA-mediated depolarizations (43-47). Under these conditions, use of substances that modulate GABA function, like barbiturates and benzodiazepines can exacerbate excitatory GABA actions even further (45). Therefore, novel approaches to treating SMEI and new insights about how different compounds, neuromodulators affect neural network activity are most needed.

In the present invention, abnormally wide CA1 circuit activation was confined using the A1R agonist N6-cyclopentyladenosine. N6-cyclopentyladenosine can act by controlling glutamate release by reduce presynaptic depolarization via the activation of delayed rectifying potassium channels (GIRK) (48) and blockade of voltage-gated calcium channels. In contrast to the agents that are typically used to treat SMEI by boosting GABA, use of A1R agonist or small molecule inhibitors downstream of A1R, like adenosine kinase (49) presents an opportunity to reduce excitation. Several recent studies show that adenosine A1 receptor agonists have anti-convulsant properties in several types of epilepsy models, including spontaneous electrographic, kindling, kainate, and seizures induced with combination of hyperthermia and an A1R antagonist (30). Furthermore, there is a ketogenic diet acting through A1Rs produces anti-convulsant effects in SCN1A mutants (50-51), and A1R activation during seizures can also prevent depolarizing GABA actions (52). Thus, the known ontogeny of synaptic E/I imbalance in mSMEI presents a window of opportunity to intervene with the process early on, during the period of robust synaptic plasticity, in order to rebalance the fragile SMEI circuit and prevent epileptogenesis, potentially through modulation of A1R or its downstream targets.

### Example 14

### Rebalancing Neural Circuits to Prevent Dravet and Autism

In addition to SMEI, about a quarter of children with Dravet syndrome have autistic like features; and mutations in SCN family genes are associated with autism (53-55). Recent experimental studies with this SCN1A transgenic model show that about a third of the HET animals develop social dysfunctions (56). Growing awareness of the relationship between epilepsy and its co-morbidities, like autism (57), further accentuates how significant it is to understand basic dysfunctions in neural circuits during the period of epileptogenesis when E/I imbalance ensues and initial seizures occur.

### Example 15

### Acute Treatment with A1R Agonist Blocks FSs In Vivo

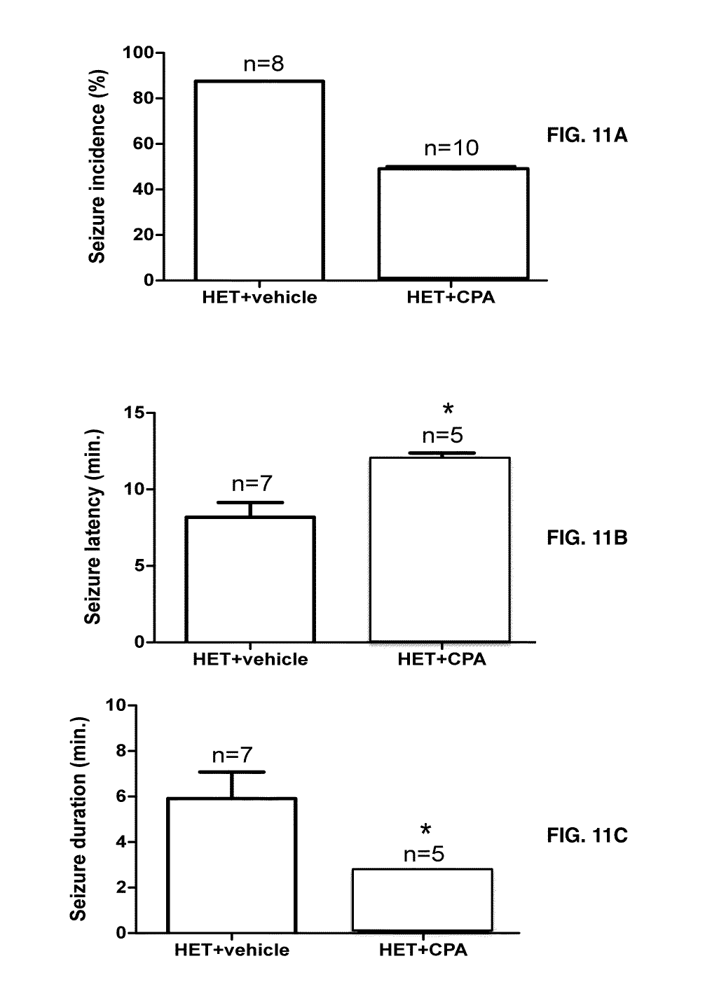

In SMEI mice pretreated with CPA (n=10) 15 minutes before induction of hyperthermia, the incidence of the hyperthermia induced FSs decreased from 87.5% to 50% and seizure latency increased significantly (8.19±0.95 min VS 11.68±0.70 min, p<0.05), while the duration was shorter (5.91±1.17 min VS 2.26±0.43 min, p<0.05) as compared to SMEI mice treated with vehicle (n=8; FIGS. 11A-11C).

### Example 16

### Effect of Chronic CPA Treatment on FS In Vivo

The A1R agonist CPA has an acute effect on the control of hyperexcitable neural network, hyperthermia-induced seizure in vitro and in vivo. To investigate if the chronic treatment of mSMEI with CPA can have long-term effects on the rewiring of the neural circuit and FS threshold activity, CPA was injected into the SMEI mice twice a day for continuous 10 days during the critical activity-dependent period of development (P11-P20), and the effects were determined 24 hours and 10 days after the last injection.

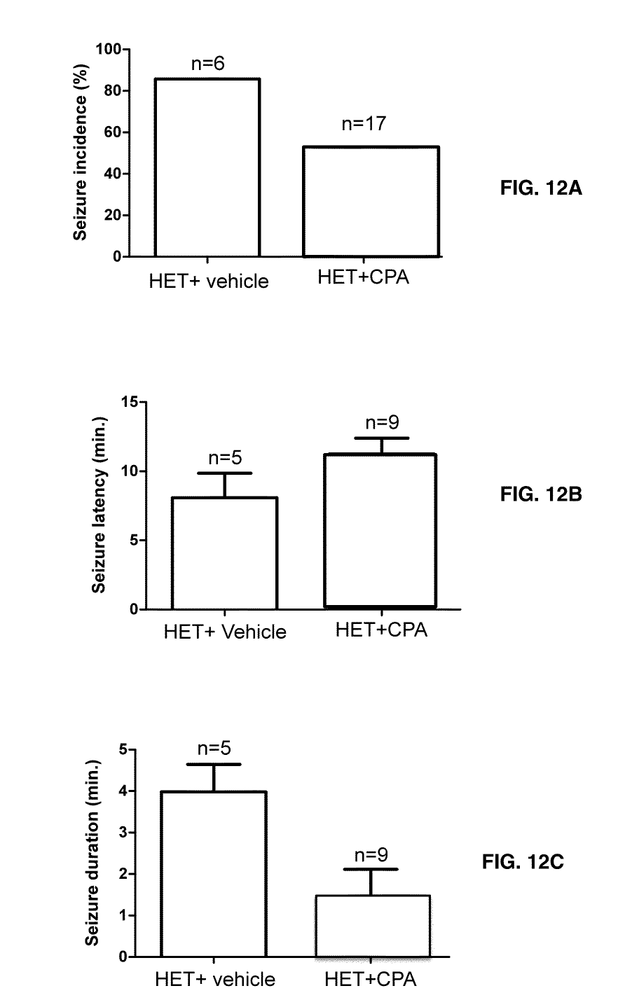

For the effect of CPA 24 hours after the last injection, compared to the vehicle group (treated with 0.9% saline), the mice treated with CPA had an increased threshold to the hyperthermia-induced seizures, as indicated by a lower seizure incidence rate (HET+vehicle: 83.3%, n=17; HET+CPA: 53%), shorter duration ((HET+vehicle: 3.980±0.6651; HET+CPA: 1.424±0.6904), and longer latency (HE+vehicle: 8.080±1.772; HET+CPA: 11.25±1.140). (FIGS. 12A-12C.)

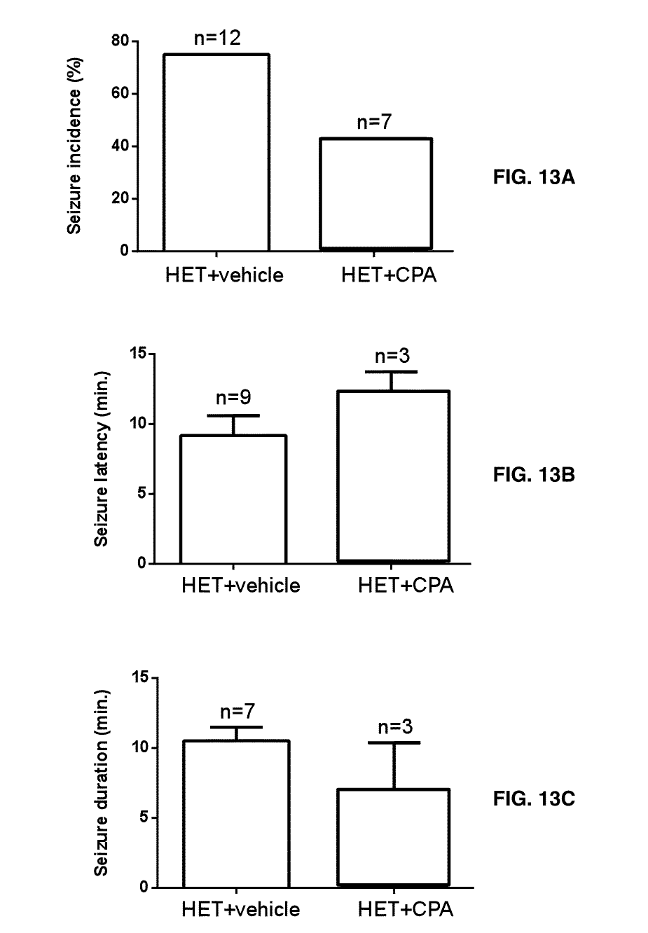

Even 10 FIG. after the last injection, the CPA treated group (different mice) demonstrated the compound's efficacy in controlling hyperthermia-induced seizures. CPA decreased the seizure incidence from 75% to 43%. Because of the limited number of CPA treated SMEI mice that developed seizures during hyperthermia, there was no significant difference between CPA group and vehicle group in terms of the seizure latency and duration. However, there is still a clear tendency toward an increase in seizure latency and a decrease in duration (FIGS. 13A-13C.

### Chronic CPA Treatment Reduces Excitation

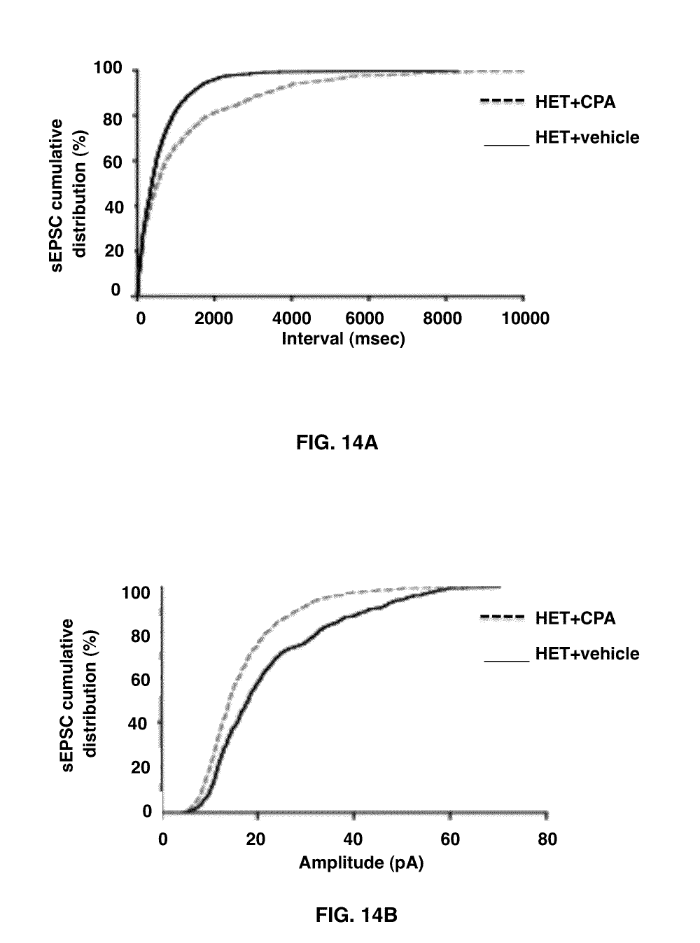

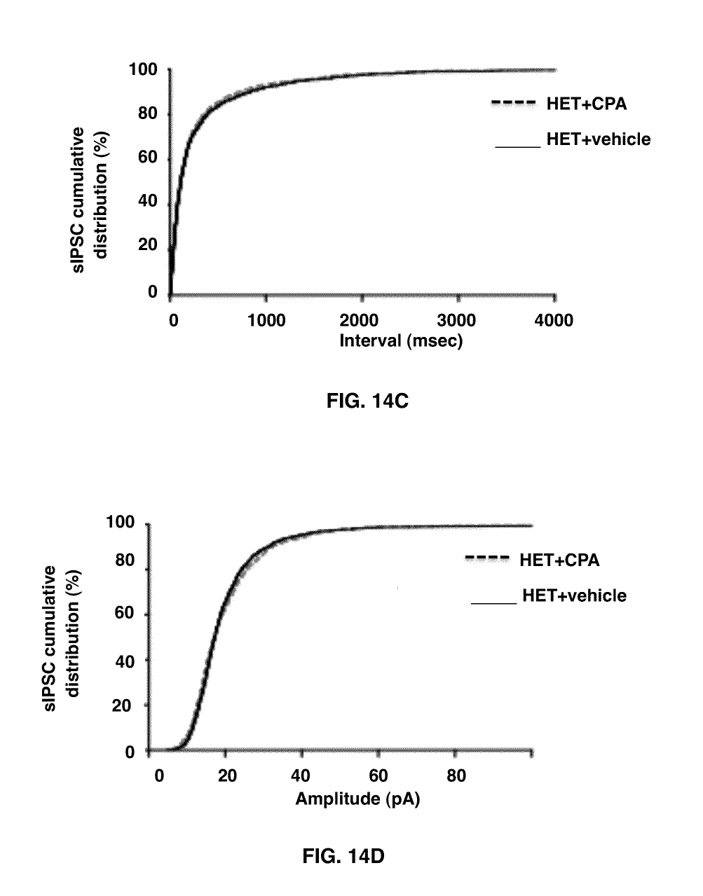

To explore how repeated CPA treatment during the critical developing period (P11-P20) exerts its long-term effect on seizure control, we investigated the E/I activity was investigated by measuring sIPSCs and sEPSCs again to see if E/I imbalance improves after chronic CPA treatment. Our results showed that chronic CPA treatment reduces both the amplitude and the frequency of sEPSCs (FIGS. 14A-14B) significantly, but it does not cause significant change in sIPSCs. (FIGS. 14C-14D).

The present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered or modified and all such variations are considered within the scope and spirit of the present invention. Also, the terms in the claims have their plain, ordinary meaning unless otherwise explicitly and clearly defined by the patentee.

The following references were cited herein:
1. Dravet et al., (2005) Adv Neurol 95:71-102.
2. Akiyama et al., (2010) Epilepsia 51:1043-1052.
3. Dravet C. (2011) Epilepsia 52 Suppl 2:3-9.
4. Guerrini R, Falchi M. (2011) Dev Med Child Neurol 53 Suppl 2:11-15.
5. Ragona F. (2011) Epilepsia 52 Suppl 2:39-43.
6. Scheffer et al., (2009) Brain Dev 31:394-400.
7. Wolff et al., (2006) Epilepsia 47 Suppl 2:45-48.
8. Catterall et al., (2010) J Physiol 588:1849-1859.
9. Escayg A, Goldin A L. (2010) Epilepsia 51:1650-1658.
10. Oakley et al., (2011) Epilepsia 52 Suppl 2:59-61.
11. Volkers et al., (2011) Eur J Neurosci 34:1268-1275.
12. Fukuma et al., (2004) Epilepsia 45:140-148.
13. Sugawara et al., (2003) Epilepsy Res 54:201-207.
14. Ogiwara et al., (2007) J Neurosci 27:5903-5914.
15. Yu et al., (2006) Nat Neurosci 9:1142-1149.
16. Chiron C, Dulac O. (2011) Epilepsia 52 Suppl 2:72-75.
17. Hayashi et al., (2011) Epilepsia 52:1010-1017.
18. Grinvald A, Hildesheim R. (2004) Nat Rev Neurosci 5:874-885.
19. Tominaga et al., (2000) J Neurosci Methods 102:11-23.
20. Zecevic et al., (2003) Curr Protoc Neurosci Chapter 6:Unit 6 17.
21. Bandyopadhyay et al., (2007) J Neurophysiol 97:4120-4128.
22. Coulter et al., (2011) J Physiol 589:1893-1903.
23. Hazra et al., (2012) J Neurophysiol.
24. Mennerick et al., (2010) J Neurosci 30:2871-2879.
25. Petersen et al., (2003) J Neurosci 23:1298-1309.
26. Abbott L F, Regehr W G. (2004) Nature 431:796-803.
27. Baxter et al., (2011) Eur J Neurosci 34:213-220.
28. O'Mara et al., (2000) Hippocampus 10:447-456.
29. Zhou F W, Roper S N. (2012) Epilepsia 53:850-859.
30. Boison D. (2010) Open Neurosci J 4:93-101.
31. Gouder N, Fritschy J M, Boison D. (2003) Epilepsia 44:877-885.
32. Masino et al., (2001) In Abbrachio M, (ed) Purinergic and Pyrimidinergic signaling. Springer-Verlag.
33. Hargus et al., (2009) Brain Res 1280:60-68.
34. Migita et al., (2008) J Neurosci Res 86:2820-2828.
35. Somjen G. (2004) Ions in the Brain. Oxford University Press, New York.
36. Yizhar et al., (2011) Nature 477:171-178.
37. Dulla C G, Huguenard J R. (2009) Nat Neurosci 12:959-960.
38. Rasband M N. (2010) Neurosci Lett 486:101-106.
39. Ang et al., (2006) J Neurosci 26:11850-11856.
40. Pinto et al., (2005) J Neurosci 25:8131-8140.
41. Ziburkus et al. (2006) J Neurophysiol 95:3948-3954.
42. Oakley et al., (2009) Proc Natl Acad Sci USA 106:3994-3999.
43. Brumback et al., (2008) J Neurosci 28:1301-1312.
44. Dzhala et al., (2010) J Neurosci 30:11745-11761.
45. Staley K. (1992) Neurosci Lett 146:105-107.
46. Staley K J, Mody I. (1992) J Neurophysiol 68:197-212.
47. Staley K J, Proctor W R. (1999) J Physiol 519 Pt 3:693-712.
48. Takigawa T, Alzheimer C. (2002) J Physiol 539:67-75.
49. Boison D. (2008) Prog Neurobiol 84:249-262.
50. Dutton et al., (2011) Epilepsia 52:2050-2056.
51. Masino et al., (2011) J Clin Invest 121:2679-2683.
52. Hie et al., (2012) J Neurosci 32:5321-5332.
53. Kamiya et al., (2004) J Neurosci 24:2690-2698.
54. O'Roak et al., (2011) Nat Genet 43:585-589.
55. Weiss et al., (2003) Mol Psychiatry 8:186-194.
56. Li et al., (2011) Epilepsy Behav 21:291-295.
57. Bolton et al., (2011) Br J Psychiatry 198:289-294.
58. Boison et al., (1999) Exp Neurol 160:164-174.
59. Fukuda et al., (2010) Epilepsia 51:483-487.
60. Guttinger et al., (2005) Exp Neurol 193:53-64.

What is claimed is:

1. A method of controlling hippocampal neural circuit hyperexcitability occurring in a neurological disease or disorder associated with epileptogenesis in a subject in need of such treatment, comprising the step of:
contacting the hippocampus in said subject with a compound effective to restore excitatory/inhibitor balance thereby controlling the neural circuit hyperexcitability.

2. The method of claim 1, wherein said compound is selected from the group consisting of adenosine, an adenosine mimetic, an adenosine modulator, an adenosine transport inhibitor and an adenosine receptor agonist.

3. The method of claim 2, wherein said adenosine receptor agonist is a adenosine receptor congener, N6-cyclopentyladenosine, N6-cyclohexyladenosine, 2-chloro-cyclopentyladenosine, N-(3(R))-tetrahydrofuranyl)-6-aminopurine riboside, or a nucleoside transporter.

4. The method of claim 2, wherein said adenosine transport inhibitor is dipyridamole, nitrobenzylthioinosine, dilazep, benzodiazepine, dihydropyridies, xanthine or quinoline derivatives.

5. The method of claim 2, wherein said adenosine modulator is selected from the group consisting of an ecto-5′-nucleotidase inhibitor, an adenosine kinase inhibitor, a S-adenosylhomocysteine hydrolase inhibitor, and an adenosine diaminase inhibitor.

6. The method of claim 1, wherein said subject suitable for selection is one with intractable epilepsy, Dravet's syndrome, febrile seizures, autism spectrum disorder or attention deficit hyperactivity disorder.

7. The method of claim 1, further comprising the step of administering a GABA modulating composition, an anticonvulsant agent, an ion channel inactivator, or a combination thereof.

8. The method of claim 7, wherein the GABA-modulating composition is selected from the group consisting of barbiturates, benzodiazepines, Gabapentin, Pregabalin, 4-aminobutanoic acid (GABA), 4-amino-3-(4-chlorophenyl)butanoic acid (baclofen), 4-amino-3-phenylbutanoic acid, 4-amino-3-hydroxybutanoic acid, 4-amino-3-(4-chlorophenyl)-3-hydroxyphenylbutanoic acid, 4-amino-3-(thien-2-yl)butanoic acid, 4-amino-3-(5-chlorothien-2-yl)butanoic acid, 4-amino-3-(5-bromothien-2-yl)butanoic acid, 4-amino-3-(5-methylthien-2-yl)butanoic acid, 4-amino-3-(2-imidazolyl)butanoic acid, 4-guanidino-3-(4-chlorophenyl)butanoic acid, (3-aminopropyl)phosphonous acid, (4-aminobut-2-yl)phosphonous acid, sodium butyrate, (3-amino-2-methylpropyl)phosphonous acid, (3-aminobutyl)phosphonous acid, (3-amino-2-(4-chlorophenyl)propyl)phosphonous acid, (3-amino-2-(4-chlorophenyl)-2-hydroxypropyl)phosphonous acid, (3-amino-2-(4-fluorophenyl)propyl)phosphonous acid, (3-amino-2-phenylpropyl)phosphonous acid, (3-amino-2-hydroxypropyl)phosphonous acid, (E)-(3-aminopropen-1-yl)phosphonous acid, (3-amino-2-cyclohexylpropyl)phosphonous acid, (3-amino-2-benzylpropyl)phosphonous acid, [3-amino-2-(4-methylphenyl)propyl]phosphonous acid, [3-amino-2-(4-trifluoromethylphenyl)propyl]phosphonous acid, [3-amino-2-(4-methoxyphenyl)propyl]phosphonous acid, [3-amino-2-(4-chlorophenyl)-2-hydroxypropyl]phosphonous acid, (3-aminopropyl)methylphosphinic acid, (3-amino-2-hydroxypropyl)methylphosphinic acid, (3-aminopropyl)(difluoromethyl)phosphinic acid, (4-aminobut-2-yl)methylphosphinic acid, (3-amino-1-hydroxypropyl)methylphosphinic acid, (3-amino-2-hydroxypropyl)(difluoromethyl)phosphinic acid, (E)-(3-aminopropen-1-yl)methylphosphinic acid, (3-amino-2-oxo-propyl)methylphosphinic acid, (3-aminopropyl)hydroxymethylphosphinic acid, (5-aminopent-3-yemethylphosphinic acid, (4-amino-1,1,1-trifluorobut-2-yl)methylphosphinic acid, (3-amino-2-(4-chlorophenyl)propyl)sulfinic acid, and 3-aminopropylsulfinic acid.

9. A method of treating a neurological disease or disorder associated with epileptogenesis in a subject in need of such treatment, comprising the step of:
administering an amount of an adenosine A1 agonist pharmacologically effective to block epileptogenetic activities without blocking excitatory synaptic transmission.

10. The method of claim 9, wherein said adenosine agonist is selected from the group consisting of adenosine, an adenosine mimetic, an adenosine modulator, an adenosine transport inhibitor and an adenosine receptor agonist.

11. The method of claim 9, wherein said adenosine receptor agonist is a adenosine receptor congener, N6-cyclopentyladenosine, N6-cyclohexyladenosine, 2-chloro-cyclopentyladenosine, N-(3(R))-tetrahydrofuranyl)-6-aminopurine riboside, or a nucleoside transporter.

12. The method of claim 9, wherein said adenosine transport inhibitor is dipyridamole, nitrobenzylthioinosine, dilazep, benzodiazepine, dihydropyridies, xanthine or quinoline derivatives.

13. The method of claim 9, wherein said adenosine modulator is selected from the group consisting of an ecto-5′-nucleotidase inhibitor, an adenosine kinase inhibitor, a S-adenosylhomocysteine hydrolase inhibitor, and an adenosine diaminase inhibitor.

14. The method of claim 9, wherein said neurological disease or disorder associated with epileptogenesis is intractable epilepsy, Dravet's syndrome, febrile seizures, autism spectrum disorder or attention deficit hyperactivity disorder.

15. The method of claim 9, further comprising the step of administering a GABA modulating composition, an anticonvulsant agent, an ion channel inactivator, or a combination thereof.

16. The method of claim 15, wherein the GABA-modulating composition is selected from the group consisting of barbiturates, benzodiazepines, Gabapentin, Pregabalin, 4-aminobutanoic acid (GABA), 4-amino-3-(4-chlorophenyl)butanoic acid (baclofen), 4-amino-3-phenylbutanoic acid, 4-amino-3-hydroxybutanoic acid, 4-amino-3-(4-chlorophenyl)-3-hydroxyphenylbutanoic acid, 4-amino-3-(thien-2-yl)butanoic acid, 4-amino-3-(5-chlorothien-2-yl)butanoic acid, 4-amino-3-(5-bromothien-2-yl)butanoic acid, 4-amino-3-(5-methylthien-2-yl)butanoic acid, 4-amino-3-(2-imidazolyl)butanoic acid, 4-guanidino-3-(4-chlorophenyl)butanoic acid, (3-aminopropyl)phosphonous acid, (4-aminobut-2-yl)phosphonous acid, sodium butyrate, (3-amino-2-methylpropyl)phosphonous acid, (3-aminobutyl)phosphonous acid, (3-amino-2-(4-chlorophenyl)propyl)phosphonous acid, (3-amino-2-(4-chlorophenyl)-2-hydroxypropyl)phosphonous acid, (3-amino-2-(4-fluorophenyl)propyl)phosphonous acid, (3-amino-2-phenylpropyl)phosphonous acid, (3-amino-2-hydroxypropyl)phosphonous acid, (E)-(3-aminopropen-1-yl)phosphonous acid, (3-amino-2-cyclohexylpropyl)phosphonous acid, (3-amino-2-benzylpropyl)phosphonous acid, [3-amino-2-(4-methylphenyl)propyl]phosphonous acid, [3-amino-2-(4-trifluoromethylphenyl)propyl]phosphonous acid, [3-amino-2-(4-methoxyphenyl)propyl]phosphonous acid, [3-amino-2-(4-chlorophenyl)-2-hydroxypropyl]phosphonous acid, (3-aminopropyl)methylphosphinic acid, (3-amino-2-hydroxypropyl)methylphosphinic acid, (3-aminopropyl)(difluoromethyl)phosphinic acid, (4-aminobut-2-yl)methylphosphinic acid, (3-amino-1-hydroxypropyl)methylphosphinic acid, (3-amino-2-hydroxypropyl)(difluoromethyl)phosphinic acid, (E)-(3-aminopropen-1-yl)methylphosphinic acid, (3-amino-2-oxo-propyl)methylphosphinic acid, (3-aminopropyl)hydroxymethylphosphinic acid, (5-aminopent-3-yemethylphosphinic acid, (4-amino-1,1,1-trifluorobut-2-yl)methylphosphinic acid, (3-amino-2-(4-chlorophenyl)propyl)sulfinic acid, and 3-aminopropylsulfinic acid.

17. A method of treating severe myoclonic epilepsy in a subject in a subject in need of such treatment, comprising the step of:
administering an amount of an adenosine A1 agonist pharmacologically effective to treat said severe myoclonic epilepsy.

18. The method of claim 17, wherein said adenosine A1 agonist is selected from the group consisting of adenosine, an adenosine mimetic, an adenosine modulator, an adenosine transport inhibitor and an adenosine receptor agonist.

19. The method of claim 17, wherein said adenosine receptor agonist is a adenosine receptor congener, N6-cyclopentyladenosine, N6-cyclohexyladenosine, 2-chloro-cyclopentyladenosine, N-(3(R))-tetrahydrofuranyl)-6-aminopurine riboside, or a nucleoside transporter.

20. The method of claim 17, wherein said adenosine transport inhibitor is dipyridamole, nitrobenzylthioinosine, dilazep, benzodiazepine, dihydropyridies, xanthine or quinoline derivatives.

21. The method of claim 17, wherein said adenosine modulator is selected from the group consisting of an ecto-5′-nucleotidase inhibitor, an adenosine kinase inhibitor, a S-adenosylhomocysteine hydrolase inhibitor, and an adenosine diaminase inhibitor.

22. The method of claim 17, further comprising the step of administering a GABA modulating composition, an anticonvulsant agent, an ion channel inactivator, or a combination thereof.

23. The method of claim 17, wherein the GABA-modulating composition is selected from the group consisting of barbiturates, benzodiazepines, Gabapentin, Pregabalin, 4-aminobutanoic acid (GABA), 4-amino-3-(4-chlorophenyl)butanoic acid (baclofen), 4-amino-3-phenylbutanoic acid, 4-amino-3-hydroxybutanoic acid, 4-amino-3-(4-chlorophenyl)-3-hydroxyphenylbutanoic acid, 4-amino-3-(thien-2-yl)butanoic acid, 4-amino-3-(5-chlorothien-2-yl)butanoic acid, 4-amino-3-(5-bromothien-2-yl)butanoic acid, 4-amino-3-(5-methylthien-2-yl)butanoic acid, 4-amino-3-(2-imidazolyl)butanoic acid, 4-guanidino-3-(4-chlorophenyl)butanoic acid, (3-aminopropyl)phosphonous acid, (4-aminobut-2-yl)phosphonous acid, sodium butyrate, (3-amino-2-methylpropyl)phosphonous acid, (3-aminobutyl)phosphonous acid, (3-amino-2-(4-chlorophenyl)propyl)phosphonous acid, (3-amino-2-(4-chlorophenyl)-2-hydroxypropyl)phosphonous acid, (3-amino-2-(4-fluorophenyl)propyl)phosphonous acid, (3-amino-2-phenylpropyl)phosphonous acid, (3-amino-2-hydroxypropyl)phosphonous acid, (E)-(3-aminopropen-1-yl)phosphonous acid, (3-amino-2-cyclohexylpropyl)phosphonous acid, (3-amino-2-benzylpropyl)phosphonous acid, [3-amino-2-(4-methylphenyl)propyl]phosphonous acid, [3-amino-2-(4-trifluoromethylphenyl)propyl]phosphonous acid, [3-amino-2-(4-methoxyphenyl)propyl]phosphonous acid, [3-amino-2-(4-chlorophenyl)-2-hydroxypropyl]phosphonous acid, (3-aminopropyl)methylphosphinic acid, (3-amino-2-hydroxypropyl)methylphosphinic acid, (3-aminopropyl)(difluoromethyl)phosphinic acid, (4-aminobut-2-yl)methylphosphinic acid, (3-amino-1-hydroxypropyl)methylphosphinic acid, (3-amino-2-hydroxypropyl)(difluoromethyl)phosphinic acid, (E)-(3-aminopropen-1-yl)methylphosphinic acid, (3-amino-2-oxo-propyl)methylphosphinic acid, (3-aminopropyl)hydroxymethylphosphinic acid, (5-aminopent-3-yemethylphosphinic acid, (4-amino-1,1,1-trifluorobut-2-yl)methylphosphinic acid, (3-amino-2-(4-chlorophenyl)propyl)sulfinic acid, and 3-aminopropylsulfinic acid.
